(19) United States
(12) Patent Application Publication
(10) Pub. No.: US 2019/0162725 A1
(43) Pub. Date: May 30, 2019

(54) MATERIALS AND METHODS RELATING TO ANTIBODY TARGETS FOR TUBERCULOSIS SEROLOGY

(71) Applicants: Mitch Magee, Chandler, AZ (US); Joshua LaBaer, Chandler, AZ (US); Lusheng Song, Tempe, AZ (US); Garrick Wallstrom, Mesa, AZ (US); Jacqueline Achkar, Bronx, NY (US)

(72) Inventors: Mitch Magee, Chandler, AZ (US); Joshua LaBaer, Chandler, AZ (US); Lusheng Song, Tempe, AZ (US); Garrick Wallstrom, Mesa, AZ (US); Jacqueline Achkar, Bronx, NY (US)

(21) Appl. No.: 16/204,674

(22) Filed: Nov. 29, 2018

Related U.S. Application Data
(60) Provisional application No. 62/592,237, filed on Nov. 29, 2017.

(51) Int. Cl. G01N 33/569

(52) U.S. Cl. CPC G01N 33/5695; G01N 2333/35; G01N 2800/26

(57) ABSTRACT

Embodiments of the present disclosure relate generally to detection and diagnosis of tuberculosis (TB) infections. More particularly, the present disclosure provides novel immunogenic biomarkers associated with TB infections for the diagnosis and treatment of TB. Novel TB biomarkers can enhance the diagnosis and treatment of TB by providing greater accuracy and efficiency, especially for point-of-care technologies.

### CROSS-REFERENCE TO RELATED APPLICATIONS

This application claims the benefit of U.S. Provisional Patent Application No. 62/592,237, filed on Nov. 29, 2017, the entire content of which is fully incorporated herein by reference.

### GOVERNMENT FUNDING

This invention was made with government support under Federal Grant Nos. R01 AI096213, AI05684, R01 AI117927, and K23 AI067665, awarded by the National Institutes of Health (NIH). The government has certain rights to this invention.

### FIELD

Embodiments of the present disclosure relate generally to detection and diagnosis of tuberculosis (TB) infections. More particularly, the present disclosure provides novel immunogenic biomarkers associated with TB infections for the diagnosis and treatment of TB.

### BACKGROUND

Active tuberculosis (TB) is a disease caused by uncontrolled infection with Mycobacterium tuberculosis (Mtb). It predominantly affects the respiratory tract and is typically transmitted through infectious droplets generated by coughing. The disease remains a major global public health problem, ranking alongside HIV infection as the leading cause of death worldwide. In 2015, an estimated 10.4 million new cases occurred globally with around 1.4 million TB-associated deaths; numbers that for the first time in decades reflect an increase in incident cases compared with the preceding year. Rapid TB diagnosis and treatment are cornerstones of TB control and essential for reduction of morbidity, mortality and transmission.

Antibody (Ab) detection assays can be adapted for the development of rapid and inexpensive tests that require neither laboratory infrastructure nor specific training. Prior serological tests for the diagnosis of TB have been insufficiently sensitive and specific for several reasons. Importantly, the Ab profiles of TB patients are heterogeneous, and tests that are based on a limited number of antigens, often only one or two, are insufficient to capture the diversity of TB cases. For example, a strong Ab response to the 38 kDa protein is elicited almost exclusively in the subgroup of advanced, HIV negative pulmonary TB patients, so assays based on this antigen are limited in diagnostic scope. Furthermore, several antigens appear to lack specificity for TB. Because of the potential to turn Ab detection assays into simple dipstick formats, TB serology, despite its known limitations, remains a field of study that is worthwhile pursuing further and new biomarker targets need to be identified. The simultaneous use of multiple more recently identified Mtb proteins in form of multiplex microbead immunoassays has already shown promising improved accuracy for TB serodiagnosis in regional case-control studies. Although the World Health Organization recognizes the limitations of currently available serologic tests and in fact cautions against using them, it vigorously encourages further research to meet the need for reliable, simple tests for TB in endemic regions. Because Ab detection is amenable to use in dipstick format incorporating a diversity of antigens, the pursuit of Ab targets that are valid biomarkers of TB is worthwhile.

Discovery of potential biomarkers requires high-throughput methods for assessing proteome-wide screens for antibody reactivity. The utilization of in situ protein arrays provides advances in the access of high-throughput protein microarray and their translation studies. Instead of requiring purified protein for printing, the in situ protein microarray utilizes printing of expression plasmids encoding libraries of genes. After in situ transcription and translation the proteins “self-assemble” on the array surface with the aid of ribosomes and chaperones, thereby enhancing natural protein folding and post-translational modification. Among the in situ protein microarray methods, Nucleic Acid Programmable Protein Array (NAPPA) represents a platform for the biomarker discovery in cancer, autoimmune diseases, and infectious disease. Membrane proteins express and display well with NAPPA with an efficiency that exceeds 90%. Because membrane proteins comprise a large portion of antigens eliciting a human humoral immune response to TB, this method could identify novel valuable Ab targets for TB serodiagnosis that might not be discovered with the conventional protein array platform that is based on printing prefabricated proteins, typically generated in E. coli, on glass slides.

Diagnosis of TB can be challenging because the clinical presentations are manifold and dependent on the immune status of the host. Furthermore, the differential diagnosis can be broad with diagnostic confirmation desired. The gold standard tests for detecting Mtb, usually in a respiratory sample, are culture or nucleic acid amplification (NAA) both of which require a certain degree of laboratory infrastructure and/or equipment, which are often not available in endemic settings, which are typically resource-limited. Thus, there is an urgent need for simple point-of-care (POC) TB tests that are based on the use of easily accessible, nonsputum based body fluids, such as blood, and that can detect the different forms of TB, pulmonary and extrapulmonary, in various hosts. In the absence of such POC tests, a simple triage method to identify those symptomatic TB suspects that are in need of further confirmatory testing, would be desirable but remains a further unmet need among the current TB diagnostic armamentarium.

### SUMMARY

Embodiments of the present disclosure relate generally to detection and diagnosis of tuberculosis (TB) infections. More particularly, the present disclosure provides novel immunogenic biomarkers associated with TB infections for the diagnosis and treatment of TB.

Embodiments of the present disclosure include a method of diagnosing a subject as having a TB infection. In accordance with these embodiments, the method includes performing an assay on a biological sample obtained from a subject, and measuring or detecting at least one TB biomarker or fragment thereof selected from the group consisting of Rv0054 (ssb), Rv0813c, Rv2031c, (HspX/acr), Rv0222 (echA1), Rv0948c, Rv2853 (PE_PGRS48), Rv3405c, and Rv3544c (fadE28). Measurement or detection of the at least one TB biomarker or fragment thereof can indicate that the subject has a TB infection.

Embodiments of the present disclosure also include a panel of biomarkers for diagnosing a subject as having a TB infection. In accordance with these embodiments, the panel includes at least one TB biomarker or fragment thereof selected from the group consisting of Rv0054 (ssb), Rv0813c, Rv2031c, (HspX/acr), Rv0222 (echA1), Rv0948c, Rv2853 (PE_PGRS48), Rv3405c, and Rv3544c (fadE28), wherein the measurement or detection of the at least one TB biomarker or fragment thereof indicates that the subject has a TB infection.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-1D include representative schematics describing the strategy underlying the identification of the TB biomarkers disclosed herein, which includes screening (FIG. 1A), deconvolution (FIG. 1B), validation (FIG. 1C), and verification (FIG. 1D).

FIGS. 2A-2D include representative images of a multiplex HD-NAPPA platform used to screen candidate genes (FIGS. 2A-2B), and the deconvolution analysis of these candidate genes (FIGS. 2A-DC) on a single gene HD-NAPPA.

FIGS. 3A-3B include representative Venn diagrams of validated IgG (FIG. 3A) and IgA (FIG. 3B) hit distribution across the four subgroups (US/HIV−; US/HIV+; SA/HIV−; SA/HIV+).

FIGS. 4A-4D include representative plots of RAPID ELISA verification of 8 selected TB biomarkers across the four subgroups (US/HIV− (FIG. 4A); US/HIV+(FIG. 4B); SA/HIV− (FIG. 4C); and SA/HIV+(FIG. 4D)).

FIGS. 5A-5D include representative ROC plots for TB diagnostic panels developed for each subgroup (US/HIV− (FIG. 5A); US/HIV+(FIG. 5B); SA/HIV− (FIG. 5C); and SA/HIV+(FIG. 5D)).

FIGS. 6A-6C include representative schematics and graphs of the multiplex HD-NAPPA platform used to probe Mtb for novel TB biomarkers, including an array (FIG. 6A), a dotplot (FIG. 6B), and a heat map (FIG. 6C).

FIGS. 7A-7D include representative schematics and graphs of quality control evaluation of Mtb M-HD-NAPPA, including an array (FIG. 7A), a dotplot (FIG. 7B), and Pearson correlation plots (FIGS. 7C and 7D).

FIGS. 8A-8D include representative schematics and graphs of quality control evaluation of Mtb M-HD-NAPPA used in deconvolution and validation analysis, including an array (FIG. 8A), a dotplot (FIG. 8B), and Pearson correlation plots (FIGS. 8C and 8D).

FIGS. 9A-9B include representative plots of Mtb protein RAPID ELISA quality control analysis, including protein expression of the 8 selected TB biomarkers (FIG. 9A), and serum responses to negative controls (FIG. 9B).

FIGS. 10A-10I include representative ROC analysis according to subgroups for candidate Mtb proteins tested via RAPID ELISA (US/HIV−: Rv0831c (FIG. 10A) and Rv2031c (FIG. 10B); US/HIV+: Rv0054 (FIG. 10C) and Rv3405c (FIG. 10D); SA/HIV−: Rv2031c (FIG. 10E), Rv0054 (FIG. 10F); Rv0831c (FIG. 10G) and Rv3405c (FIG. 10H); and SA/HIV+: Rv3405c (FIG. 10I)).

FIGS. 11A-11B include representative schematics and graphs of quality control evaluation of Mtb protein expression on NAPPA, including an array (FIG. 11A), and dotplots (FIG. 11B).

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

“Isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. [NA “Polypeptide” and “isolated polypeptide” as used herein refers to a polymer of amino acids or amino acid derivatives that are connected by peptide bonds. An isolated polypeptide is a polypeptide that is isolated from a source. An isolated polypeptide can be at least 1% pure, at least 5% pure, at least 10% pure, at least 20% pure, at least 40% pure, at least 60% pure, at least 80% pure, and at least 90% pure, as determined by one or more protein biochemistry techniques (e.g., SDS-PAGE).

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. The subject or patient may be undergoing other forms of treatment.

“Treat,” “treated,” or “treating,” as used herein, refer to a therapeutic method wherein the object is to slow down (lessen) an undesired physiological condition, disorder or disease, or to obtain beneficial or desired clinical results. In some aspects of the present disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment also includes prolonging survival as compared to expected survival if not receiving treatment.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” is used herein to describe a nucleic acid molecule that can transport another nucleic acid to which it has been linked. One type of vector is a “plasmid”, which refers to a circular double-stranded DNA loop into which additional DNA segments may be ligated. Another type of vector is a viral vector, wherein additional DNA segments may be ligated into the viral genome. Certain vectors can replicate autonomously in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. “Plasmid” and “vector” may be used interchangeably as the plasmid is the most commonly used form of vector. However, other forms of expression vectors, such as viral vectors (e.g., replication defective retroviruses, adenoviruses and adeno-associated viruses), which serve equivalent functions, can be used. In this regard, RNA versions of vectors (including RNA viral vectors) may also find use in the context of the present disclosure.

Before any embodiments of the present disclosure are explained in detail, it is to be understood that the present disclosure is not limited in its application to the details of construction and the arrangement of components set forth in the following description or illustrated in the accompanying drawings. The present disclosure is capable of other embodiments and of being practiced or of being carried out in various ways.

Other aspects of the invention will become apparent by consideration of the detailed description and accompanying drawings.

Embodiments of the present disclosure include the generation of a novel Mtb protein microarray based on the NAPPA platform, the High Density-NAPPA (HD-NAPPA), which was used in a multiplex version (M-HD-NAPPA) for high-throughput screening and in a single protein version for deconvolution and validation. This platform entailed printing plasmids containing cDNAs encoding the Mtb proteome comprising ˜4000 proteins into silicon nano-wells. In accordance with these embodiments, sera from HIV uninfected and coinfected TB patients and controls from different geographic regions (US and South Africa (SA)) were screened and proteins with previously unknown value for TB serodiagnosis were identified.

Embodiments of the present disclosure include the generation and validation of a new whole-proteome Mtb HD-NAPPA and demonstrate its value for detecting novel biomarkers for TB serodiagnosis. Embodiments of the present disclosure demonstrate the feasibility, efficiency, and accuracy of multiplexing proteins into a single spot for expedited high-throughput screening for Ab responses to the Mtb proteome. In accordance with these embodiments, multimarker panels were established to distinguish TB patients from non-infected or latently infected subjects, with and without HIV coinfection across two geographic regions. With this initial evaluation, 8 proteins were identified that show potential as TB diagnostic biomarkers. HD-NAPPA provides a higher signal to noise ratio for Ab biomarker discovery as compared with flat-glass NAPPA. Using M-HDNAPPA arrays, where the screen utilizes multiplexing of targets, can further accelerate Ab biomarker screening. To perform serum Ab profiling over the whole Mtb proteome (around 4000 genes), flat-glass based NAPPA requires two slides per sample. In contrast, HD-NAPPA requires only a half slide and the M-HD-NAPPA (using 3-target multiplex per spot) requires only a quarter of a slide. Thus, the capacity to process 8 times more samples than flat-glass based NAPPA would not only facilitate the Ab discovery speed, but also result in significant reagent cost savings.

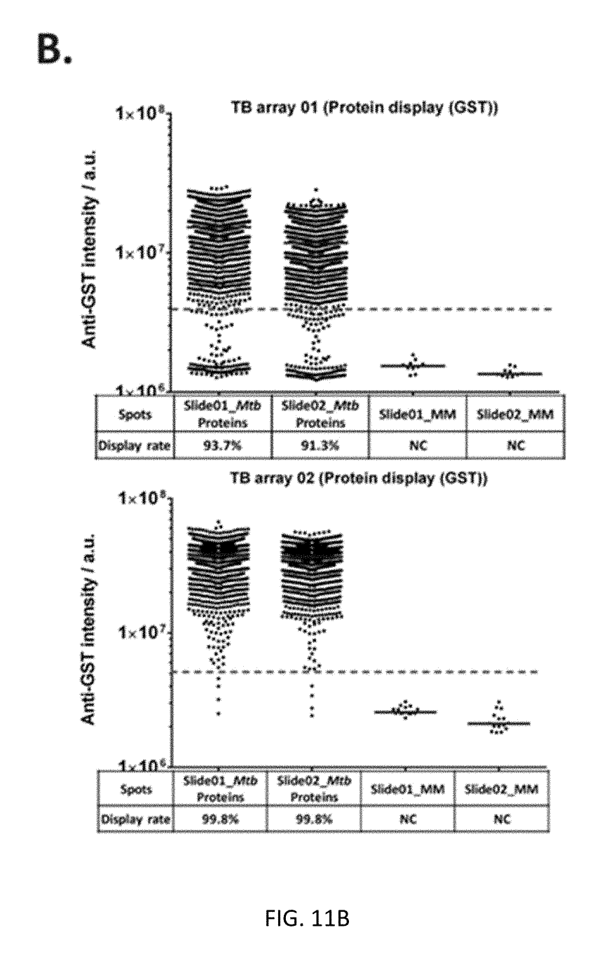

The possibility that low protein expression levels, from one of the three proteins in the mix, could mask detection was analyzed. Results showed that 100% of high and medium signal intensity responses and 91.5% of low-signal intensity responses were detected when proteins were mixed in all possible combinations. Printing 4,045 plasmids required creating two glass arrays, termed TB array 01 and TB array 02. With these arrays of individually-printed Mtb plasmids, expression of Mtb proteins was demonstrated by detection of the fusion partner with anti-GST staining. As shown in FIG. 11, TB array 01 and TB array 02 contained 4,045 Mtb proteins and the display rate for TB array 01 were from 91.3% to 93.7% whereas the TB array 02 were 99.8% separately and the overall display rates were 96.1%.

It was also investigated whether some or all of the observed regional differences in Ab responses are driven by regional differences in disease state—with TB patients from resource-limited TB endemic settings typically being diagnosed at more advanced stages than those living in the US, or whether the regional differences could be driven in part by infection with different Mtb strains. Embodiments of the present disclosure therefore included individual panels for the four subject subgroups, depending on the geographic region (US or SA) and HIV status (HIV+/−). The eight candidate immunoreactive Mtb proteins identified have varied characteristics. Four of these proteins are secreted and have been identified in Mtb culture filtrates (CFPs; Rv0054, Rv0831c, Rv2031c and Rv0222), with three of these (Rv0054, Rv0831c, Rv2031c) also identified in the cell membrane and two (Rv0831c, Rv2031c) in the cell wall. One (Rv0948c) has only been associated with the Mtb membrane fraction. The cellular location for two of the proteins (Rv3405c and Rv3544c) has not been identified.

Embodiments of the present disclosure include a method of diagnosing a subject as having a TB infection. In accordance with these embodiments, the method includes performing an assay on a biological sample obtained from a subject, and measuring or detecting at least one TB biomarker or fragment thereof selected from the group consisting of Rv0054 (ssb), Rv0813c, Rv2031c, (HspX/acr), Rv0222 (echA1), Rv0948c, Rv2853 (PE_PGRS48), Rv3405c, and Rv3544c (fadE28). Measurement or detection of the at least one TB biomarker or fragment thereof can indicate that the subject has a TB infection.

In some embodiments, a biological sample includes a fluid sample from a subject having or suspected of having TB. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample may be assayed directly. In a particular example, the biological sample is a human bodily substance (e.g., bodily fluid, blood such as whole blood, serum, plasma, urine, saliva, sweat, sputum, semen, mucus, lacrimal fluid, lymph fluid, amniotic fluid, interstitial fluid, lung lavage, cerebrospinal fluid, feces, tissue, organ, or the like). Tissues may include, but are not limited to skeletal muscle tissue, liver tissue, lung tissue, kidney tissue, myocardial tissue, brain tissue, bone marrow, cervix tissue, skin, and the like. The sample may be a liquid sample or a liquid extract of a solid sample. In certain cases, the source of the sample may be an organ or tissue, such as a biopsy sample, which may be solubilized by tissue disintegration/cell lysis. In some embodiments, the biological sample is at least one of whole blood, serum, plasma, urine, saliva, sweat, sputum, semen, mucus, lacrimal fluid, lymph fluid, amniotic fluid, interstitial fluid, lung lavage, cerebrospinal fluid, and feces.

In some embodiments, methods of diagnosing a subject as having or not having TB can be carried out using any suitable diagnostic test, such as but not limited to, an immunoassay. Methods of determining the presence or amount (detecting or measuring) a TB biomarker include, but are not limited to, immunoassays, such as sandwich immunoassays (e.g., monoclonal-monoclonal sandwich immunoassays, monoclonal-polyclonal sandwich immunoassays, including enzyme detection (enzyme immunoassay (EIA) or enzyme-linked immunosorbent assays (ELISA), competitive inhibition immunoassays (e.g., forward and reverse), enzyme multiplied immunoassay techniques (EMIT), a competitive binding assay, bioluminescence resonance energy transfer (BRET), one-step antibody detection assays, homogeneous assays, heterogeneous assays, capture on the fly assay, and the like.

In some embodiments, assays used to measure or detect a TB biomarker as described herein can be associated with percentages of sensitivity and specificity. Sensitivity of an assay as used herein refers to the proportion of subjects for whom the outcome is positive that are correctly identified as positive. Specificity of an assay as used herein refers to the proportion of subjects for whom the outcome is negative that are correctly identified as negative. In some embodiments, the immunoassay has a sensitivity of at least 80.0% and a specificity of at least 50.0%. In other embodiments, the immunoassay has a specificity of at least 80.0% and a sensitivity of at least 50.0%.

Embodiments of the present disclosure also include a panel of biomarkers for diagnosing a subject as having a TB infection. In accordance with these embodiments, the panel includes at least one TB biomarker or fragment thereof selected from the group consisting of Rv0054 (ssb), Rv0813c, Rv2031c, (HspX/acr), Rv0222 (echA1), Rv0948c, Rv2853 (PE_PGRS48), Rv3405c, and Rv3544c (fadE28), wherein the measurement or detection of the at least one TB biomarker or fragment thereof indicates that the subject has a TB infection.

A biomarker panel can refer to a set of biomarkers that can be used alone, together, or in subcombinations to indicate the status of a human subject with respect to a condition, status, or state of being of the human subject. The biomarkers within the panel of biomarkers can include those TB biomarkers discussed herein. It will be appreciated that the specific identity of biomarkers within the panel and the number of distinct biomarkers within the panel can depend on the particular use to which the biomarker panel is put and the stringency that the results of panel must meet for the particular application. In some embodiments, a TB biomarker panel can include TB biomarkers, such as those described here, as well as other various biomarkers that may or may not be used to measure or detect a TB biomarker. In some embodiments, the biomarker panel may include 2, 3, 4, 5, 6, 7, 8, 9, 10, 11, 12, 13, 14, 15 or as many as 40 biomarkers. In some embodiments, the biomarker panel may include 10 or fewer biomarkers, depending various factors such as the characteristics of the subject from which the biological sample was obtained and is to be assayed. In other embodiments, the biomarker panel may include 2, 3, 6 or 8 biomarkers. In some embodiments, the biomarker panel may be optimized from a candidate pool of biomarkers. By way of non-limiting example, the biomarker panel may be optimized for determining whether a subject has a specific disease, such as TB.

### EXAMPLES

The following examples are illustrative of disclosed methods. In light of the present disclosure, those of skill in the art will recognize that variations of these examples and other examples of the disclosed method would be possible without undue experimentation.

### Example 1

M-HD-NAPPA—Concept Validation.

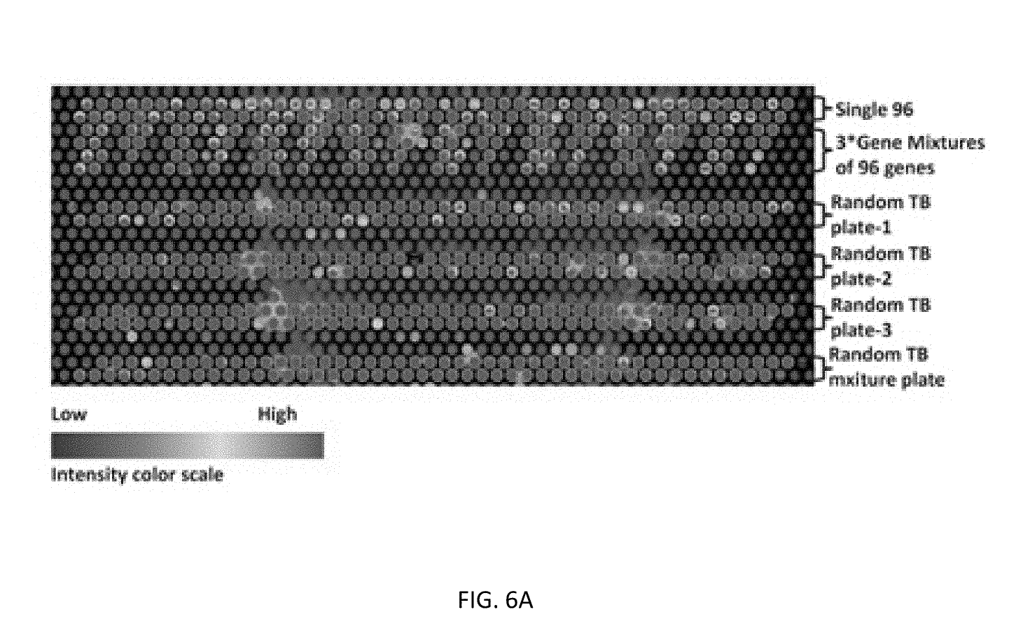

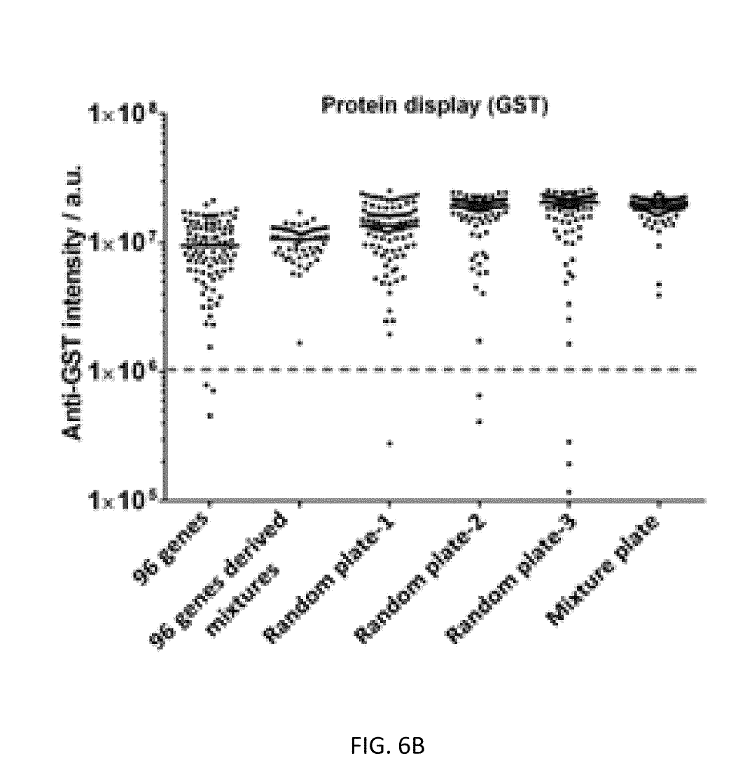

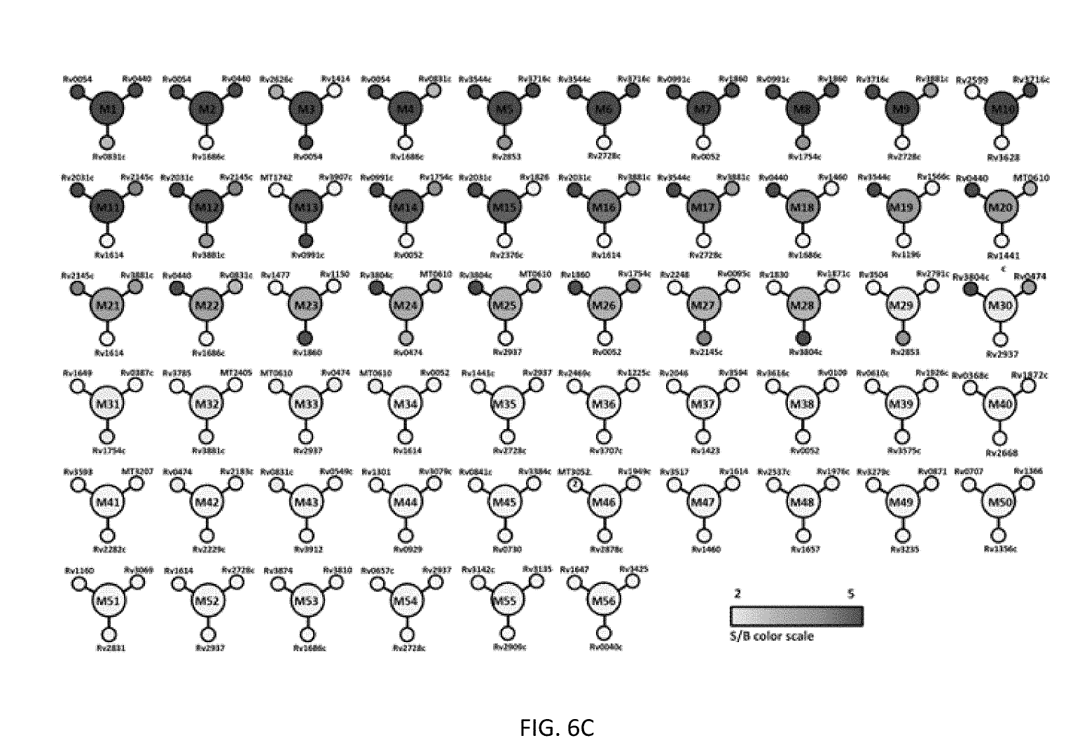

A test set of 96 Mtb proteins was first generated, identified by preliminary studies using a serum pool from TB+ patients, as well as proteins reported in TB serology literature. These test proteins were to create multiplex mixes of 3 proteins per spot to validate the concept of M-HD-NAPPA. To confirm detection of a positive responder even when it was mixed with nonresponders (i.e., that the nonresponders did not dilute the responder signal too much), it was ensured that all possible combinations of positive responders and nonresponders were created as determined by reactivity to the serum pool. To ensure that selection did not create any bias, 3 random plates were selected from the Mtb collection set to add an additional 288 Mtb genes. The gene mixtures, as well as the single genes, were printed on HD-NAPPA array. After expression, the arrays were probed with the TB+ pooled serum, sera from each of the individuals that comprise the pool, and anti-GST with signals normalized as described earlier (FIGS. 6A and 6B). The quantified signal intensities were used to establish the Signal/Background (S/B) values for evaluating spot reactivity. Using the 96 individual protein array, a tool was created to evaluate signal intensities of the three-target mix as well as the individual component responses (FIG. 6C). The S/B values of the mixed protein responses revealed that all of the three-protein mixes and their individual components were detected for the mid- and high-level reactive protein mixtures (30/30; 100.0%) and were mostly detected if including the low reactive protein mixtures (32/35; 91.5%) using this multiplex testing strategy, demonstrating that responses to positive proteins could be observed reliably when mixed with two nonreactive proteins. It was concluded that this level of reactivity of a three-target mix was satisfactory for screening purposes.

As shown in FIG. 6A, array images demonstrate protein display as assessed by anti-GST binding of both single protein and mixture of 3 proteins/spot. FIG. 6B includes dotplots of anti-GST expression intensity for both single protein and mixture of 3 proteins/spot. Greater than 96% of the proteins express at the cutoff level of 1×10⁶ fluorescent intensity units. Blue bars represent median reactivity. FIG. 6C includes a ball and stick heat map of single and multiplex protein responses to pool serum. This ball-map schematic is based on the normalized response value of a multiplex protein spots for the central circle. Each of the component three proteins orbits the central circle and their color also represents the normalized response as individual protein spot. The darker the red color indicates higher responses, with signal to background ratio (S/B) values ranging from 2.0 to >5.0. One key criteria is a minimal S/B value of 2.0 to establish a level of minimal reactivity and 16 individual proteins met this cutoff value. It was determined that 4 proteins represented low-level reactivity (S/B 2.0-3.0), 5 proteins revealed mid-level reactivity (S/B 3.0-5.0), and 7 individual proteins revealed high-level responses (S/B>5.0).

### Example 2

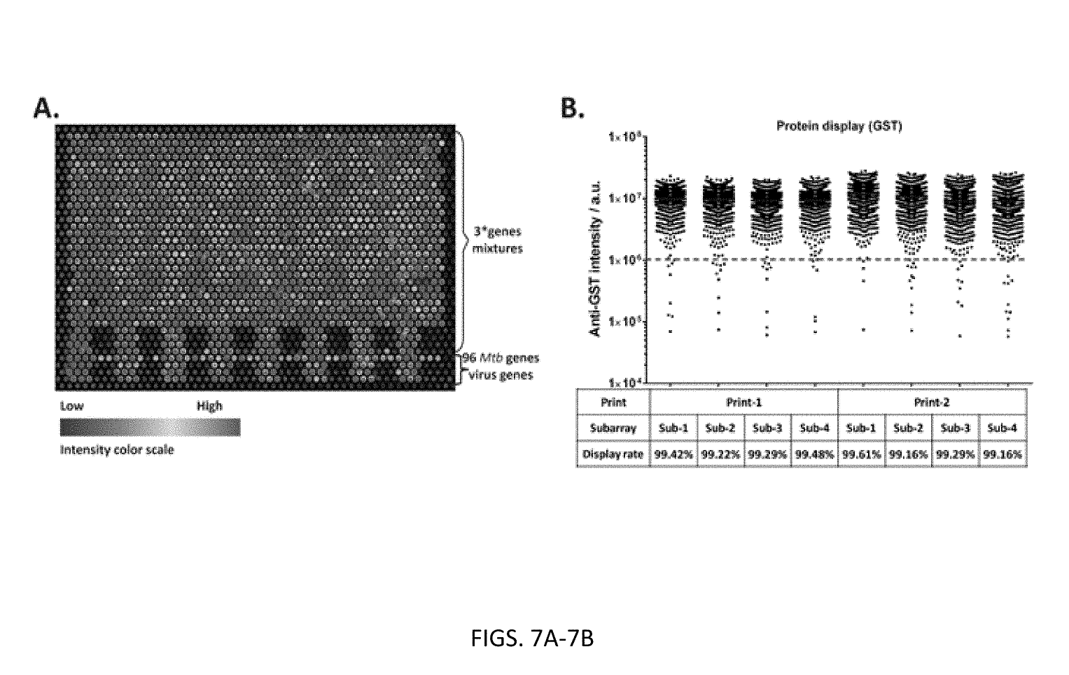

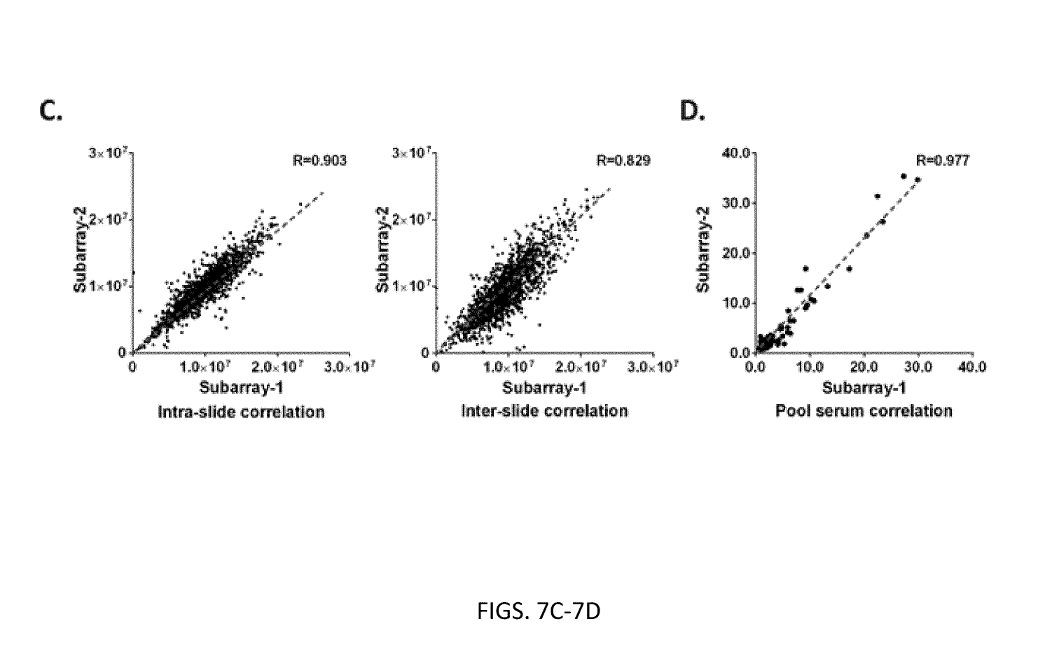

M-HD-NAPPA Screen. Following the successful demonstration of protein reactivity with M-HD-NAPPA, a protein display quality control (QC) test was performed with the 4045 Mtb M-HD-NAPPA array to assess the intra- and intersubarray correlation (protein display repeatability). The anti-GST reactivity showed that almost all of the spots exhibited a yellow to red color revealing overall fluorescence intensity higher than 1×10⁶ arbitrary intensity units (a.u., the cutoff of the successful protein display; FIGS. 7A-7B). The successful protein display rate on the M-HD-NAPPA was as high as 99.45%±0.14% across the subarrays and across two slides. The intra- and interslide correlations revealed great reproducibility (r=0.903 and r=0.829, respectively; FIG. 7C). Reproducibility testing using binding of positive control TB+ pool revealed positive interslide correlations (0.977; FIG. 7D).

FIG. 7A includes an array image of protein display as assessed by anti-GST binding to Mtb 3 protein/spot M-HD-NAPPA array. FIG. 7B includes dotplots of the protein display of full Mtb M-HD-NAPPA array across subarray and print batch. Blue bars represent median reactivity. FIG. 7C includes Pearson correlation of the anti-GST expression intensity intra- and inter-slides. FIG. 6D includes Pearson correlation of Pool serum binding to two slides of the M-HD-NAPPA array.

After assuring the quality and reproducibility of reactivity using the M-HD-NAPPA, samples were randomized to assure a mix of subject category samples during each run and day to ensure minimal run-to-run bias. All of the samples were analyzed as 4 paired subgroups according to the region (US/SA), HIV (HIV±) and TB status (TB±) for determining sensitivity and specificity. Using these groupings, spots were selected for deconvolution analysis. A representative image is shown in FIG. 2A.

FIG. 2 describes multiplex HD-NAPPA screening of candidate genes (FIGS. 2A-2B) and deconvolution of candidates on single gene HD-NAPPA (FIGS. 2C-2D). FIG. 2A is a representative sample being probed against the 1431 Mtb 3*protein spots (4,045 individual Mtb proteins) with the M-HD-NAPPA array. The array image shows color intensity from blue (no binding) to red (with very high antibody binding). Four selected binding spots on this image are highlighted, named 1-4, respectively. The inset below the array image shows a zoomed view of the four selected spots. In FIG. 2B, the median normalized serological response of the multiplex protein spots, 96 Mtb genes, the 7 viral control genes, human IgG and master mix are shown as a dot plot. The red dots in the multiplex column represent the normalized reactivity of the 4 selected spots in FIG. 2A. FIG. 2C includes a representative image of the Ab binding to the 870 single Mtb protein HD-NAPPA array. From the 4 spots in FIG. 2A, the response to 12 individual proteins (zoomed view highlighted in inset under the HD NAPPA image) can be resolved. Within each 3 protein set, 1 out of 3 proteins showed responses to serum probing. In FIG. 2D, the median normalized serological response of the single Mtb protein spots, 7 viral proteins, human IgG and master mix are shown in the dot plot. The 4 responsive proteins are highlighted in red.

Overall, 95 (US/HIV−), 47 (US/HIV+), 15 (SA/HIV−) and 30 (SA/HIV+) multispots reactive with IgG and 41 (US/HIV−), 24 (US/HIV+), 21 (SA/HIV−) and 41 (SA/HIV+) multispots reactive with IgA with more than 10% sensitivity at 70% specificity were identified. When combining all groups, 202 multispots in IgG and 144 multispots in IgA analysis showed higher than 5% sensitivity. Taken together, 163 multispots from the subgroup analysis and 259 multispots from the merged group analysis, together with 2 extra hits from visual analysis resulted in a total of 272 multispots (792 individual genes) selected for deconvolution.

### Example 3

HD-NAPPA—Deconvolution.

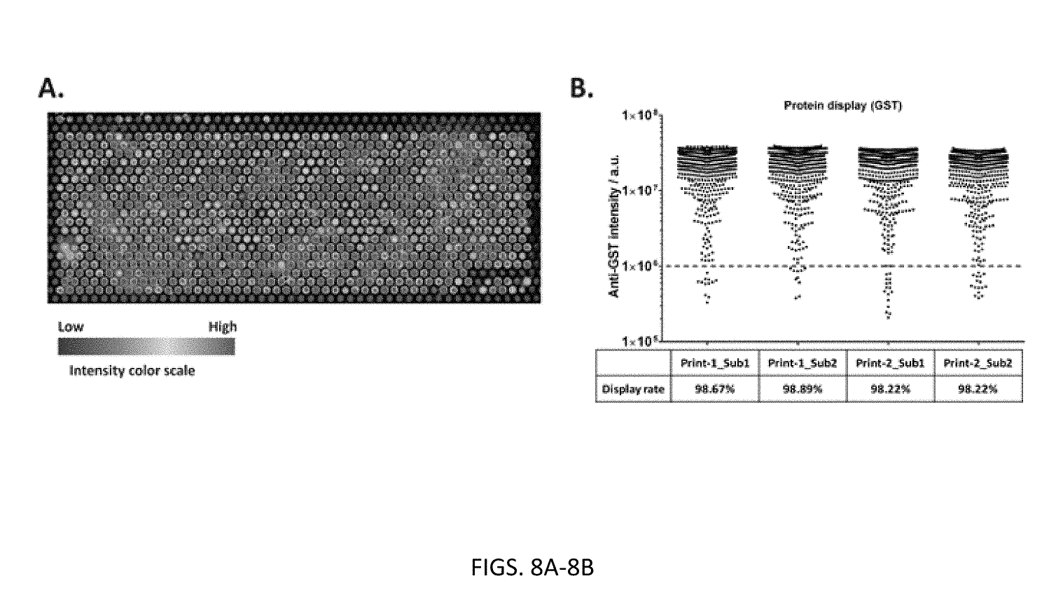

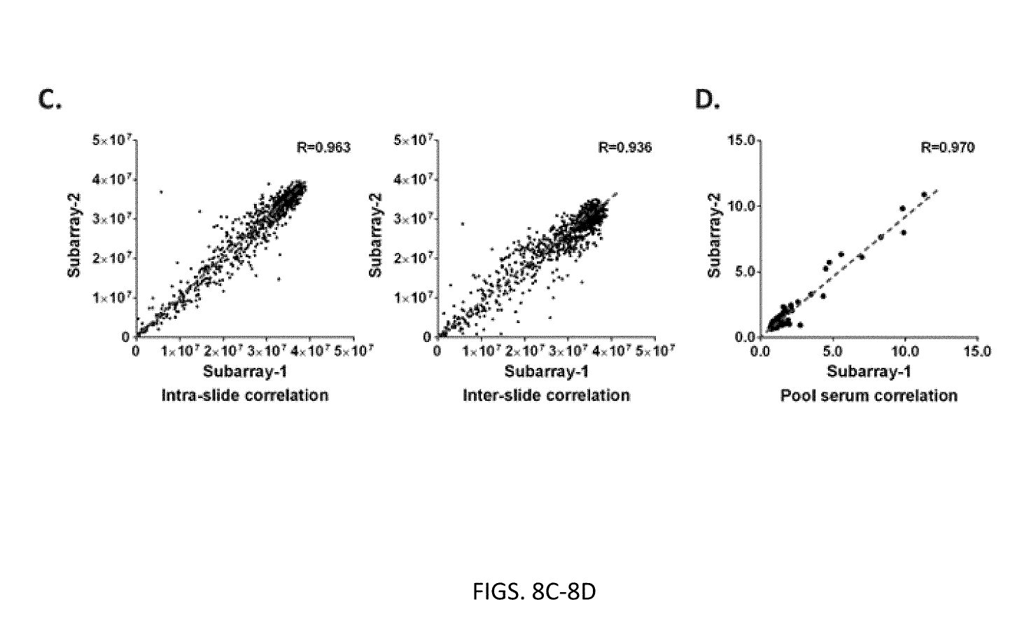

In addition to the 792 candidate proteins identified in the M-HD-NAPPA screen, the 96 Mtb proteins were included (from unpublished data and published studies) to create 870 individual arrays for printing onto HD-NAPPA. Quality control assessments of these arrays are presented in FIG. 8. These arrays were probed initially against the same samples tested in the M-HD-NAPPA discovery analysis to allow for the identification of the specific reactive proteins that were responsible for the signal at the multispots. A representative image of a subject's serum binding to the deconvolution array is presented in FIG. 2C. Three hundred and sixteen single proteins that showed higher than 10% sensitivity and 70% specificity in the subgroup analysis were selected for further validation.

FIGS. 8A-8D include quality control evaluation of Mtb HD-NAPPA used in deconvolution and validation studies. FIG. 8A includes an array image of protein display as assessed by anti-GST binding to single protein/spot Mtb HD-NAPPA array. Spots that exhibit overall fluorescence intensity higher than 1×10⁶ a.u. exceed the cutoff of the successful protein expression. FIG. 8B includes dotplots of the protein display of full Mtb HD-NAPPA array across subarray and print batch. The success of protein display rates on the multiplex HD-NAPPA were as high as 98.50%±0.34% across 4 subarrays across two slides. Blue bars represent median reactivity. In FIG. 8C, intra-slide and inter-slide Pearson correlation plots reveal great reproducibility with intra-slide subarray correlation (R=0.963) and the inter-slide correlation (R=0.936). In FIG. 8D, reproducibility testing using binding of a positive control TB+ pool revealed significant inter-slide Pearson correlations (0.970).

### Example 4

HD-NAPPA—Validation.

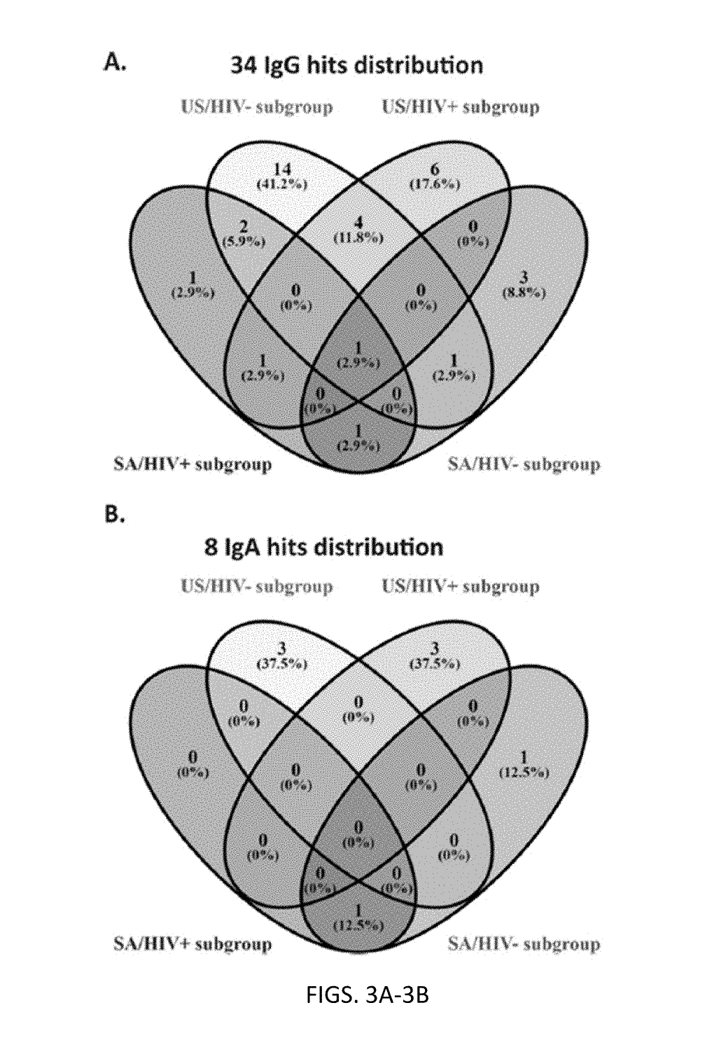

Arrays with the same genes generated for the deconvolution were also used for validation experiments with 124 biologically independent samples (Table 1). For biomarker candidate analysis, sensitivity and specificity were used as the first criteria. In addition, an odd's ratio higher than 1.5 was used; an AUC value higher than 0.55. From the combined criteria, 34 IgG hits and 8 IgA hits (FIGS. 3A-3B, respectively) were identified as potential biomarker targets with a sensitivity higher than 20%, an odd's ratio >1.5 and AUC value >0.55. The responses of these targets is shown in (Table 2) by geographic and HIV status. A combined analysis of these hits was performed with a data set that combined all of the deconvolution and validation data. Eight IgG hits (Table 3) with a sensitivity higher than 20%, an odd's ratio >1.5 and an AUC value >0.55 in the combined analysis were selected as the biomarkers for ELISA verification testing.

In FIGS. 3A-3B, validated IgG and IgA hits are presented as a Venn diagram distribution across the four subgroups. In FIG. 3A, a Venn diagram of >20% sensitivity IgG hits. In FIG. 3B, a Venn diagram of >20% sensitivity IgA hits.

TABLE 1
Sample distribution according to experiments and study phase.

| | Region | | | | | | | |
|---|---|---|---|---|---|---|---|---|
| | US | | | | South Africa | | | |
| | HIV status | | | | | | | |
| | HIV− | | HIV+ | | HIV− | | HIV+ | |
| | TB status | | | | | | | |
| | TB− | TB+ | TB− | TB+ | TB− | TB+ | TB− | TB+ |
| Multiplex HD- | 11 | 21 | 12 | 8 | 12 | 5 | 10 | 41 |
| NAPPA (n = | | | | | | | | |
| 120) | | | | | | | | |
| Deconvolution | 6 | 20 | 6 | 7 | 6 | 5 | 6 | 38 |
| (n = 94)^{a} | | | | | | | | |
| Validation (n = | 11 | 23 | 11 | 9 | 12 | 6 | 12 | 40 |
| 124)^{b} | | | | | | | | |
| ELISA (n = | 22 | 45 | 46 | 21 | 25 | 13 | 24 | 89 |
| 285)^{c} | | | | | | | | |

^{a}Because the deconvolution of positive reactions was the prime goal of this experiment, we focused these analyses predominantly on TB+ samples from the muliplex HD-NAPPA screening.
^{b}Consisting of biologically independent samples.
^{c}Consisting of the original screening and validation samples (n = 244) and heretofore untested samples (n = 41).

TABLE 2
Top IgG hits for US/HIV− (A), US/HIV+ (B),
SA/HIV− (C), and SA/HIV+ (D).

| | Cutoff-1.4 | | Odd's ratio | | AUC | |
|---|---|---|---|---|---|---|
| Protein | Sensitivity | Specificity | Value | P value | Value | P value |

A. Top IgG hits for US/HIV−

| | | | | | | |
|---|---|---|---|---|---|---|
| Rv0054 | 34.8% | 90.9% | 3.83 | 0.18 | 0.81 | 0.00 |
| Rv3544c | 34.8% | 90.9% | 3.83 | 0.18 | 0.68 | 0.05 |
| Rv1731 | 34.8% | 81.8% | 1.91 | 0.29 | 0.60 | 0.16 |
| Rv3260c | 30.4% | 100.0% | 6.09 | 0.25 | 0.64 | 0.10 |
| Rv0178 | 30.4% | 100.0% | 6.09 | 0.17 | 0.60 | 0.16 |
| Rv2770c | 30.4% | 81.8% | 1.67 | 0.53 | 0.58 | 0.23 |
| Rv2306c | 26.1% | 100.0% | 5.22 | 0.14 | 0.82 | 0.00 |
| Rv2031c | 26.1% | 100.0% | 5.22 | 0.23 | 0.73 | 0.02 |
| Rv2668 | 26.1% | 100.0% | 5.22 | 0.30 | 0.72 | 0.02 |
| Rv3897c | 26.1% | 90.9% | 2.87 | 0.25 | 0.66 | 0.06 |
| Rv0983 | 26.1% | 100.0% | 5.22 | 0.30 | 0.65 | 0.08 |
| Rv0474 | 26.1% | 100.0% | 5.22 | 0.23 | 0.62 | 0.14 |
| Rv3822 | 26.1% | 100.0% | 5.22 | 0.25 | 0.60 | 0.16 |
| MT3033 | 26.1% | 100.0% | 5.22 | 0.23 | 0.59 | 0.19 |
| Rv2831 | 21.7% | 100.0% | 4.35 | 0.23 | 0.83 | 0.00 |
| Rv3912 | 21.7% | 100.0% | 4.35 | 0.39 | 0.70 | 0.03 |
| Rv1860 | 21.7% | 100.0% | 4.35 | 0.40 | 0.65 | 0.08 |
| Rv1904 | 21.7% | 100.0% | 4.35 | 0.23 | 0.64 | 0.10 |
| Rv0040c | 21.7% | 100.0% | 4.35 | 0.40 | 0.64 | 0.10 |
| Rv2922c | 21.7% | 90.9% | 2.39 | 0.30 | 0.63 | 0.11 |

Top IgA hits for US/HIV−

| | | | | | | |
|---|---|---|---|---|---|---|
| Rv0052 | 47.8% | 81.8% | 2.63 | 0.25 | 0.72 | 0.02 |
| Rv2922c | 21.7% | 100.0% | 4.35 | 0.17 | 0.64 | 0.09 |
| Rv0509 | 21.7% | 90.9% | 2.39 | 0.23 | 0.55 | 0.31 |

B. Top IgG hits for US/HIV+

| | | | | | | |
|---|---|---|---|---|---|---|
| Rv2853 | 88.9% | 63.6% | 2.44 | 0.10 | 0.66 | 0.12 |
| MT3033 | 55.6% | 90.9% | 6.11 | 0.12 | 0.79 | 0.02 |
| Rv3810 | 44.4% | 100.0% | 8.89 | 0.08 | 0.77 | 0.02 |
| Rv0109 | 44.4% | 100.0% | 8.89 | 0.25 | 0.75 | 0.03 |
| Rv0040c | 44.4% | 72.7% | 1.63 | 0.47 | 0.68 | 0.09 |
| Rv3260c | 44.4% | 90.9% | 4.89 | 0.47 | 0.62 | 0.19 |
| Rv1748 | 33.3% | 100.0% | 6.67 | 0.13 | 0.83 | 0.01 |
| Rv3835 | 33.3% | 90.9% | 3.67 | 0.25 | 0.67 | 0.10 |
| Rv0247c | 33.3% | 100.0% | 6.67 | 0.13 | 0.63 | 0.17 |

Top IgA hits for US/HIV+

| | | | | | | |
|---|---|---|---|---|---|---|
| Rv1411c | 44.4% | 81.8% | 2.44 | 0.25 | 0.70 | 0.07 |
| Rv1754c | 33.3% | 100.0% | 6.67 | 0.48 | 0.60 | 0.24 |
| Rv0983 | 33.3% | 100.0% | 6.67 | 0.23 | 0.56 | 0.34 |

C. Top IgG hits for SA/HIV−

| | | | | | | |
|---|---|---|---|---|---|---|
| Rv2668 | 66.7% | 75.0% | 2.67 | 0.34 | 0.68 | 0.11 |
| Rv0638 | 50.0% | 83.3% | 3.00 | 0.19 | 0.85 | 0.01 |
| Rv0831c | 50.0% | 91.7% | 6.00 | 0.09 | 0.81 | 0.02 |
| Rv3405c | 50.0% | 66.7% | 1.50 | 0.24 | 0.64 | 0.17 |
| Rv1651c | 50.0% | 91.7% | 6.00 | 0.09 | 0.60 | 0.26 |

Top IgA hitsfor SA/HIV−

| | | | | | | |
|---|---|---|---|---|---|---|
| Rv1566c | 50.0% | 83.3% | 3.00 | 0.45 | 0.74 | 0.06 |

D. Top IgG hits for SA/HIV+

| | | | | | | |
|---|---|---|---|---|---|---|
| Rv3405c | 62.5% | 58.3% | 1.50 | 0.49 | 0.56 | 0.25 |
| Rv3822 | 27.5% | 83.3% | 1.65 | 0.52 | 0.58 | 0.20 |
| Rv2770c | 27.5% | 91.7% | 3.30 | 0.20 | 0.57 | 0.23 |
| Rv0652 | 20.0% | 100.0% | 4.00 | 0.20 | 0.68 | 0.03 |

Top IgA hits for SA/HIV+

TABLE 3
Candidate TB biomarkers.

| | | | | Known TB | Sensitivity | Selected |
|---|---|---|---|---|---|---|
| | Protein | Cell | | serology | estimates/ | TB serology |
| Gene | name(s) | fraction(s)^{a} | Function^{a} | antigen | comments | references |
| Rv0054 | ssb | CFP, CM | DNA recombination and repair | Yes | 12-42% | (9, 10, 41) |
| Rv0831c | Rv0831c | CFP, CM, CW, | Unknown | Yes | 25-76% | (9, 42) |
| | | cytosol | | | | |
| Rv2031c | HspX (acr) | CFP, CM, CW, | Heat shock/stress protein | Yes | 39-52% | (9, 10, 43, 44) |
| | | cytosol | induced by anoxia | | | |
| Rv0222 | echA1 | CFP | Possibly oxidizes fatty acids | Yes | 54-71% | (45, 46) |
| Rv0948c | Rv0948c | CM | Involved in the shikimate pathway | No | | |
| Rv2853 | PE_PGRS48 | Not reported | Unknown | No | | |
| Rv3405c | Rv3405c | Not reported | May be involved in transcriptional | No | | |
| | | | mechanism | | | |
| Rv3544c | fadE28 | Whole cell | Unknown | No* | | |
| | | lysates | | | | |

CFP: culture filtrate proteins; CM: cell membrane; CW: cell wall.
^{a}Information on the cellular location and functions of the proteins can be accessed through the TubercuList Database (http://tuberculist.epfl.ch/) under the gene name.

### Example 5

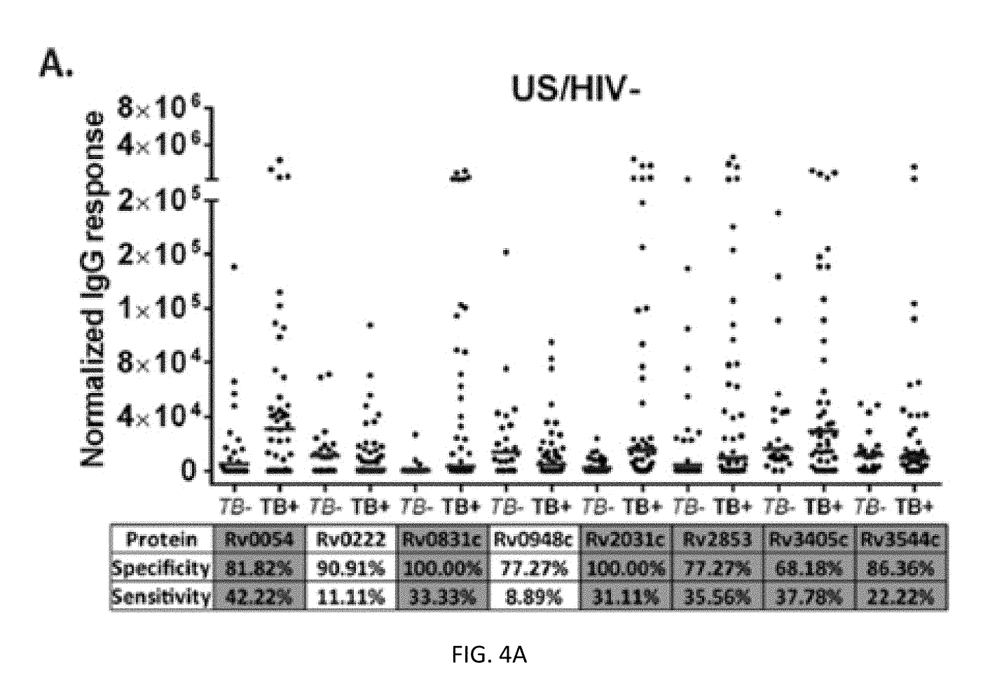

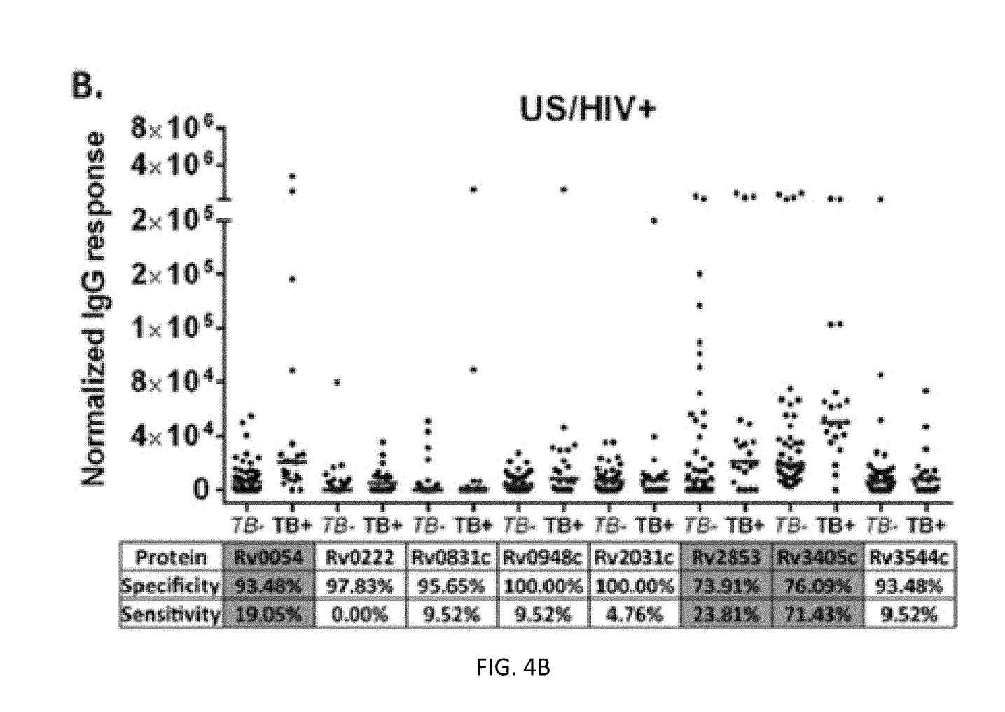

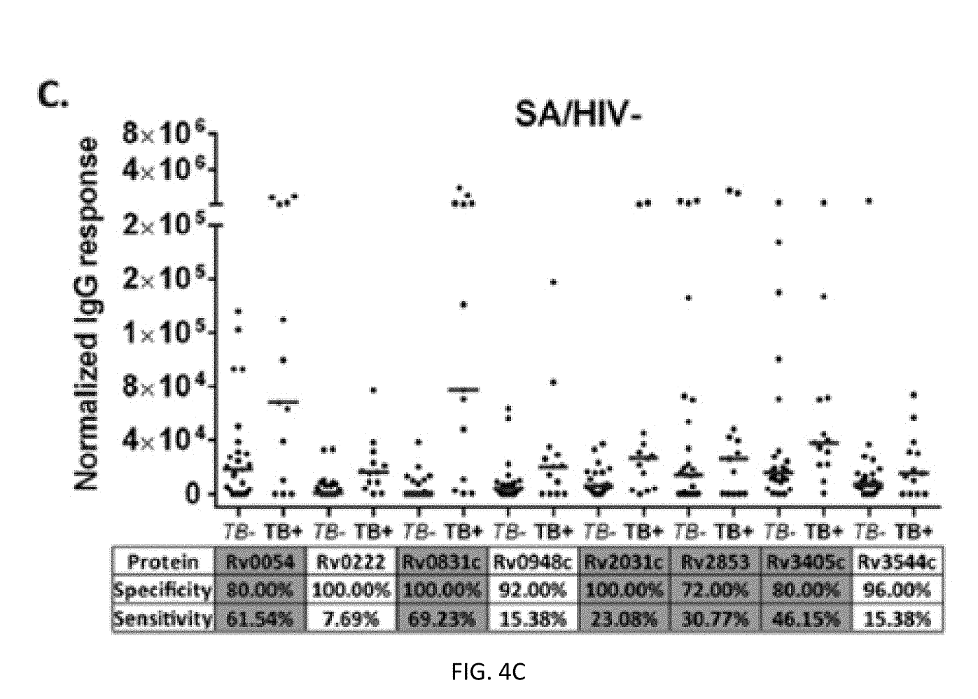

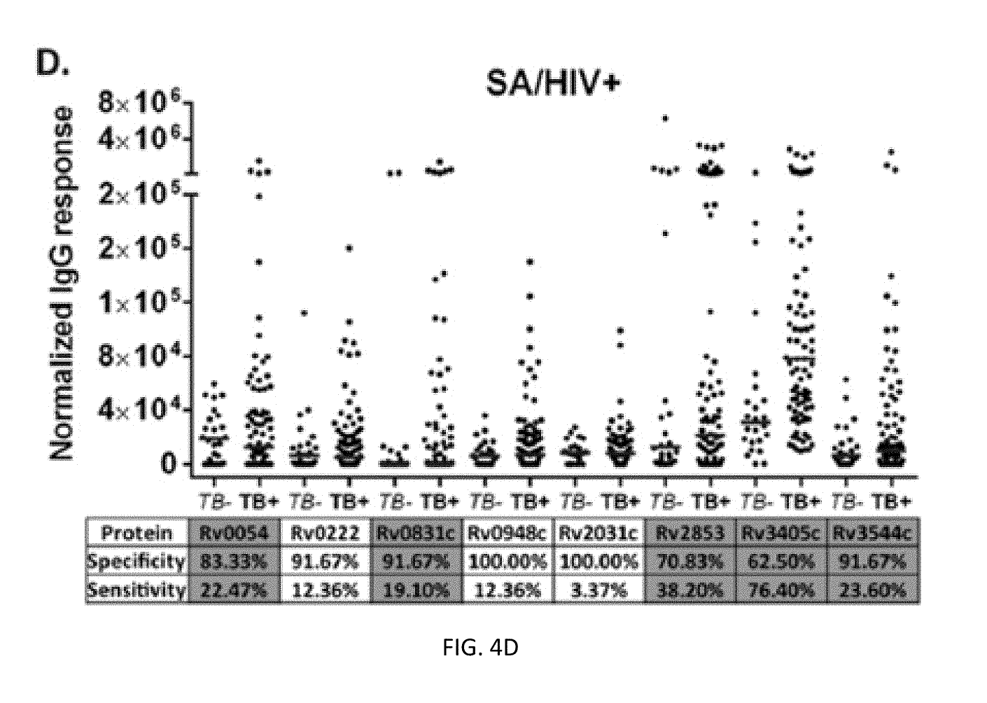

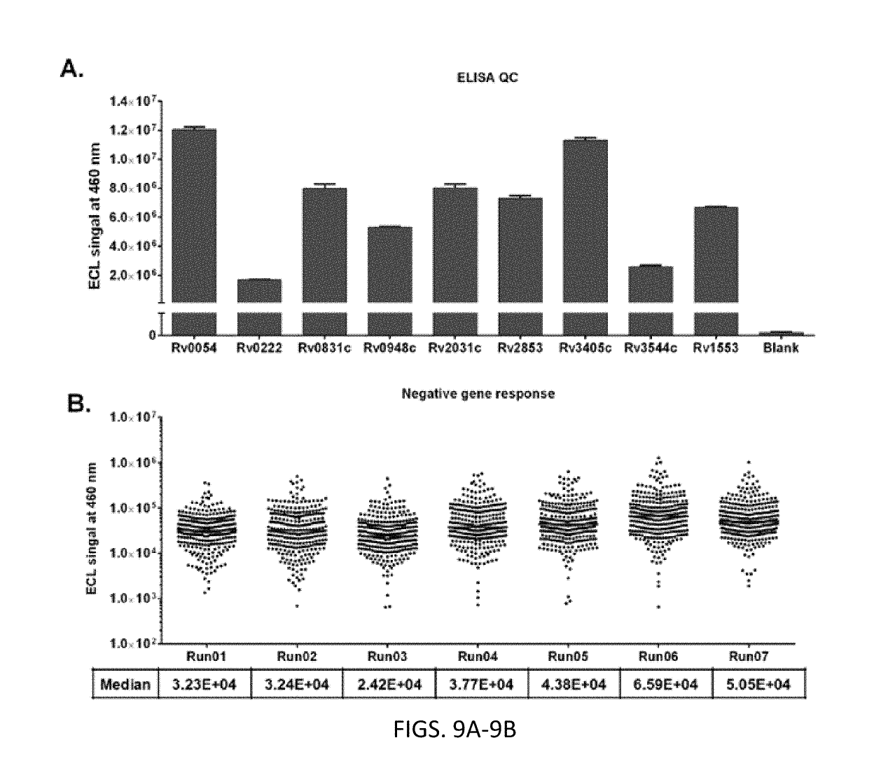

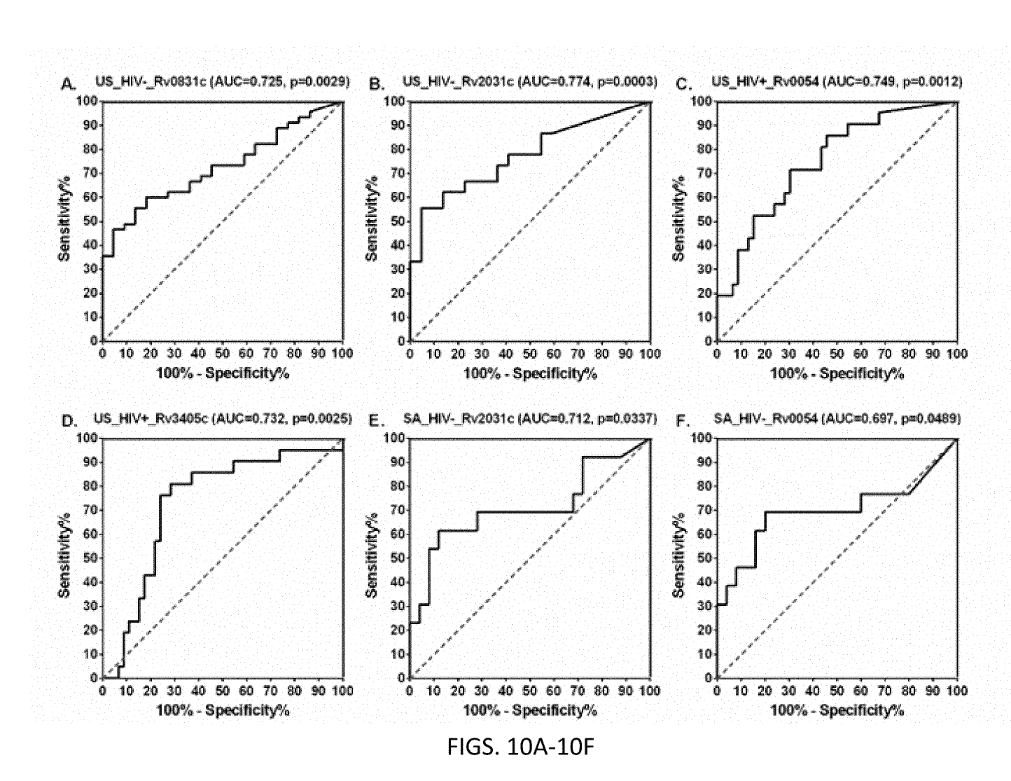

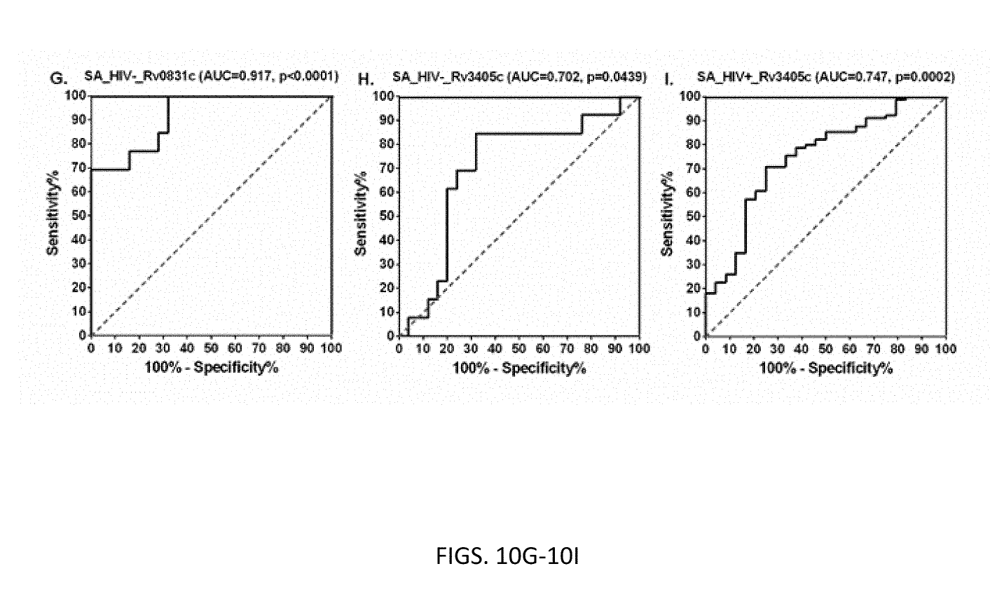

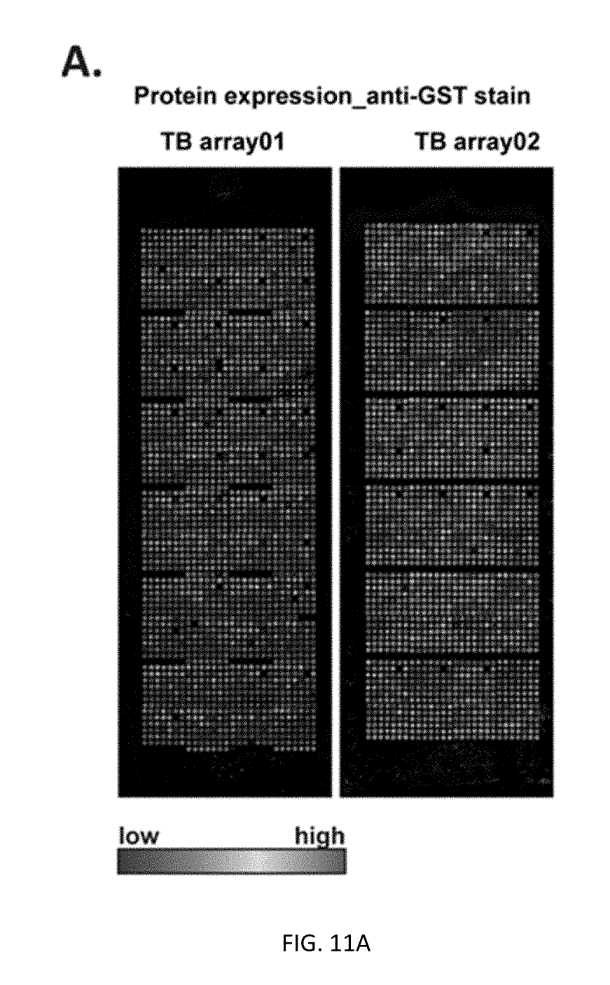

ELISA Verification. In order to verify the Mtb protein performance of the M-HD-NAPPA workflow, the rapid antigenic protein in situ display ELISA was used. All available 244 sera were tested from discovery and validation with an additional 41 samples (Table 2). An anti-GST quality control expression assessment was performed of all targets prior to performing ELISA (FIG. 9A-9B). The sensitivity and specificity of all 8 target proteins were calculated according to the four subgroups (FIG. 4). ROC analysis was also conducted and the AUC values were calculated (Table 4). There were 1-4 proteins in each subgroup that showed an AUC value of 0.7 or higher (FIG. 10). Because the specificity at fixed cutoff varied because of the heterogeneity of responses between subgroups, the sensitivity at 80% specificity and the specificity at 80% sensitivity were also evaluated (Table 4). The proteins with the highest sensitivity at 80% specificity for the four subgroups were Rv0831c (60.0%, US/HIV−), Rv0054 (52.4%, US/HIV+), Rv0831c/Rv0222 (76.9%, SA/HIV−) and Rv3405c (57.3%, SA/HIV+). The proteins with the highest specificity at 80% sensitivity for the four subgroups were Rv2031 (45.5%, US/HIV−), Rv3405c (71.7%, US/HIV+), Rv0831c (72.0%, SA/HIV−) and Rv3405c (54.2%, SA/HIV+). Although Rv0831c showed an AUC value=0.917 for the SA/HIV− subgroup, none of the other subgroups had any single protein with an AUC response above 0.9.

TABLE 4
ROC statistics for ELISA verification markers and panels.

| | | | | |
|---|---|---|---|---|
| Gene | AUC | p value | % Sens at 80% Spec | % Spec at 80% Sens |

US/HIV− (22 TB− 45 TB+)

| | | | | |
|---|---|---|---|---|
| Rv2853 | .610 | 0.1475 | 31.1 | 40.9 |
| Rv2031c | .774 | 0.0003 | 62.2 | 45.5 |
| Rv0054 | .652 | 0.0409 | 53.3 | 31.5 |
| Rv0831c | .725 | 0.0029 | 60.0 | 36.4 |
| Rv3405c | .594 | 0.2144 | 33.3 | 27.3 |
| Rv3544c | .490 | 0.8932 | 28.9 | 14.9 |
| Rv0222 | .462 | 0.6165 | 20.0 | 27.3 |
| Rv0948c | .366 | 0.0757 | 8.9 | 9.1 |
| PANEL* | .807 | <0.0001 | 64.4 | 72.7 |

US/HIV+ (46 TB− 21 TB+)

| | | | | |
|---|---|---|---|---|
| Rv2853 | .620 | 0.1169 | 14.3 | 47.8 |
| Rv2031c | .507 | 0.9238 | 14.3 | 15.7 |
| Rv0054 | .749 | 0.0011 | 52.4 | 58.5 |
| Rv0831c | .516 | 0.8393 | 19.0 | 28.3 |
| Rv3405c | .732 | 0.0025 | 42.9 | 71.7 |
| Rv3544c | .518 | 0.8162 | 28.6 | 19.8 |
| Rv0222 | .596 | 0.2108 | 47.6 | 19.6 |
| Rv0948c | .705 | 0.0074 | 47.6 | 37.0 |
| PANEL* | .782 | 0.0002 | 61.9 | 71.7 |

SA/HIV− (25 TB− 13 TB+)

| | | | | |
|---|---|---|---|---|
| Rv2853 | .546 | 0.6444 | 15.4 | 24.0 |
| Rv2031c | .712 | 0.0337 | 61.5 | 28.0 |
| Rv0054 | .697 | 0.0479 | 69.2 | 17.3 |
| Rv0831c | .917 | 0.0000 | 76.9 | 72.0 |
| Rv3405c | .702 | 0.0439 | 61.5 | 68.0 |
| Rv3544c | .622 | 0.2212 | 53.8 | 13.0 |
| Rv0222 | .831 | 0.0009 | 76.9 | 64.0 |
| Rv0948c | .705 | 0.0406 | 69.2 | 28.0 |
| PANEL* | .868 | <0.0001 | 85.3 | 84.0 |

SA/HIV− (24 TB− 89 TB+)

| | | | | |
|---|---|---|---|---|
| Rv2853 | .521 | 0.7574 | 13.5 | 12.5 |
| Rv2031c | .512 | 0.8519 | 15.7 | 20.8 |
| Rv0054 | .497 | 0.9661 | 22.5 | 14.3 |
| Rv0831c | .594 | 0.1582 | 34.8 | 41.7 |
| Rv3405c | .747 | 0.0002 | 57.3 | 54.2 |
| Rv3544c | .570 | 0.2923 | 30.3 | 16.9 |
| Rv0222 | .525 | 0.7072 | 27.0 | 20.8 |
| Rv0948c | .567 | 0.3118 | 34.8 | 20.8 |
| PANEL* | .723 | 0.0008 | 53.9 | 50.0 |

FIGS. 4A-4D describe RAPID ELISA verification of the selected 8 proteins across the four subgroups: US/HIV− (FIG. 4A), US/HIV+(FIG. 4B), SA/HIV− (FIG. 4C) and SA/HIV+(FIG. 4D). Protein sensitivity and specificity analysis under fixed cutoff=4×10⁴ arbitrary units (a.u.). Those higher than 20% sensitivity proteins were highlighted in red. Blue bars represent median normalized IgG reactivity.

FIGS. 9A-9D include data pertaining to Mtb protein RAPID ELISA quality control analysis. FIG. 9A describes protein expression of the 8 selected antigens and negative control protein (Rv1553). The target proteins were captured onto ELISA plates and the samples probed with monoclonal anti-GST and detected via chemiluminescence to assess protein capture. All 8 targets and 1 negative control proteins were detected between 2.0×10⁶ a.u. and 1.2×10⁷ a.u., which is more than 100 fold greater than the cutoff of 2.0×10⁴ a.u. (blank intensity plus two time standard deviation) and indicates good expression of the target protein. Blue bars represent the standard deviation. FIG. 9B describes serum response to negative control protein (Rv1553) across runs with an average value of median at 4.10±1.39×10⁴ a.u. with significant correlations (r>0.8;). A cutoff was then set as 4.0×10⁴ a.u. post subtracting normalization of the data, which was the median response of Rv1553 across all samples. Blue bars represent the median response.

FIGS. 10A-10I include ROC analyses according to subgroups for candidate Mtb proteins tested via RAPID ELISA. Single hit ROC analysis (AUC>0.7, p<0.05) across four subgroups: US/HIV−: Rv0831c (FIG. 10A) and Rv2031c (FIG. 10B); US/HIV+: Rv0054 (FIG. 10C) and Rv3405c (FIG. 10D); SA/HIV−: Rv2031c (FIG. 10E), Rv0054 (FIG. 10F); Rv0831c (FIG. 10G) and Rv3405c (FIG. 10H); and SA/HIV+: Rv3405c (FIG. 10I).

### Example 6

Multimarker Panels.

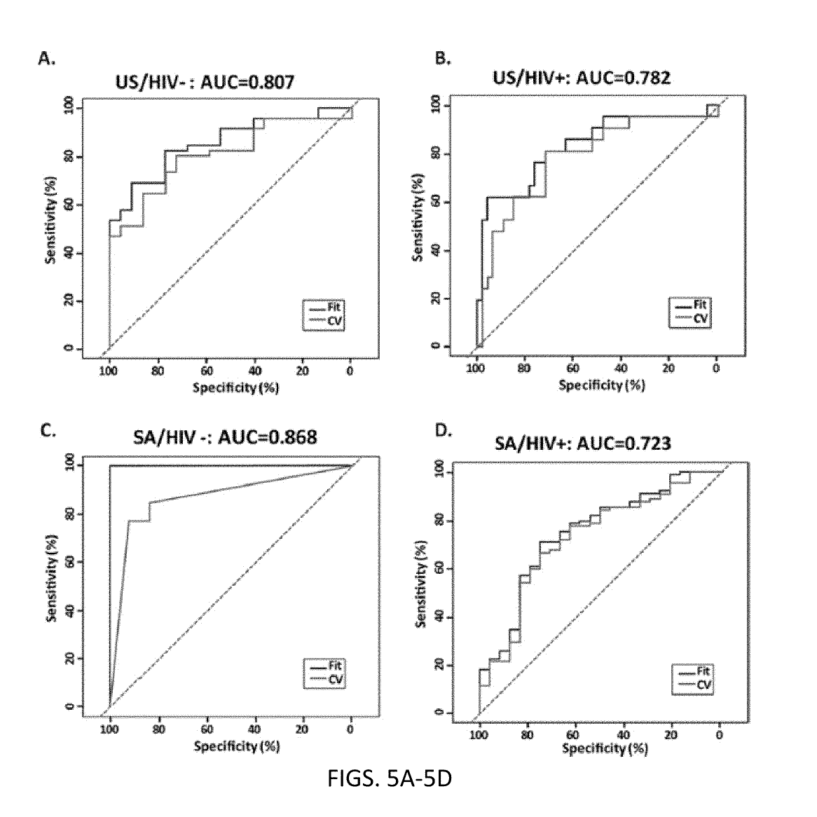

Optimal markers under BIC for the US/HIV− subgroup were Rv2031c, Rv0831c and Rv0948c. This classifier had an AUC of 0.807 under leave-one-out cross validation. For the US/HIV+ subgroup, the optimal markers were Rv0054 and Rv0948c, and the classifier had an AUC of 0.782. For the SA/HIV− subgroup, the optimal markers were Rv2853, Rv0054, Rv0831c, Rv3544c and Rv0222. The AUC under cross validation for the SA/HIV− subgroup was 0.868. Finally, for the SA/HIV+ subgroup, only Rv3405c was selected for use in the classifier, which had an AUC of 0.723 under cross validation. The ROC curves for each classifier are shown in FIG. 5 and the statistics presented in Table IV.

FIGS. 5A-5D includes ROC analysis for TB diagnostic panels developed for each subgroup. FIG. 5A includes US/HIV−: Rv2031c, Rv0831c and Rv0948c. FIG. 5B includes US/HIV+: Rv0054 and Rv0948c. FIG. 5C includes SA/HIV−: Rv2853, Rv0054, Rv0831c, Rv3544c and Rv0222. FIG. 5D includes SA/HIV+: Rv3405c. (CV: cross-validation.)

FIGS. 11A-11B include individual Mtb protein expression quality control analysis on NAPPA. FIG. 11A includes array images of individual Mtb NAPPA arrays (4,045 Mtb proteins) printed onto two glass slides and the expression with IVTT were assessed by anti-GST binding to individual protein (on the right) on the NAPPA array. FIG. 11B includes individual Mtb protein expression quantification of two of both TB array 01 and TB array 02. The TB array 01 has a display rate of 91.3% to 93.7% while the TB array 02 has a display rate of 99.8% at the cutoff of 5×10⁶ a.u. (Master mix spots (MM) plus 3 standard deviation).

### Materials and Methods

Mtb Plasmid Construction and DNA Preparation.

In the present disclosure, 3295 Mtb H37Rv and 437 CDC 1551 genes were obtained in entry vectors from the Pathogen Functional Genomics Center. Primers were designed and obtained for the missing ˜800 H37Rv genes (Integrated DNA Technologies, Coralville, Iowa) and performed PCR amplification from genomic Mtb H37Rv DNA to create entry clones for these missing genes as described. After two rounds of PCR amplification and transfer of clones to the pANT7-cGST expression vector, which encodes a C-terminal fusion partner for the target gene of Glutathione-S-Transferase (GST), a final sequence-verified gene set was obtained and was comprised of 3646 H37Rv and 399 CDC 1551 clones (4045 total) for array construction. The reduction in clone numbers resulted from failure to either produce a PCR product or creation of a verified expression clone. Purified plasmid DNA was prepared with a high throughput alkaline lysis miniprep protocol as described. For positive controls, several genes were used encoding for the antigens of the Epstein-Barr virus (EBV), a virus over 95% of individuals are infected with by adult age (22), specifically the Epstein-Barr Nuclear Antigen (EBNA), EBV Small capsomere-interacting protein (BFRF3), EBV_EBNA2, and other viral genes, specifically H1N1_Nucleoprotein, H3N2_Nucleoprotein, HCMV2_Viral transcription factor IE2 (UL122). For negative gene controls, a plasmid encoding GST without any fusion partner was used.

HD-NAPPA Array Fabrication.

The HD-NAPPA array fabrication included three main processes: nanowell slide fabrication, plasmid plate and printing mixture preparation and piezoelectric printing. The nano-well slide fabrication was performed as reported. The plasmid plate was constructed as reported for the HD-NAPPA, with modifications to the multiplex version to allow for a more high-throughput evaluation. Three unique genes were admixed into one well resulting in three unique proteins displayed in each spot. Although this added the need to deconvolute reactive spots by reassessing the same screening samples with a new microarray containing only individual proteins per spot, it allowed screening faster overall. The printing master mix (MM) was composed of polyclonal anti-GST Ab (GE Healthcare), bovine serum albumin (BSA, Sigma-Aldrich), BS3 cross linker (Pierce) and DEPC treated water. To control for secondary Ab reactivity, purified mouse IgG, human IgG and human IgA were also printed, in MM at concentrations from 40 to 200 ng/μ1 in each subarray. Negative controls consisted of MINI spots without any plasmid and the plasmid encoding only for the fusion partner GST. The HD-NAPPA print was performed on AU302 piezoelectric dispensing system (Engineering Arts LLC, Tempe, Ariz., USA) by depositing MM (1200 pL/well) and plasmid(s) (100 ng/μl, 300 pL/well) sequentially utilizing 16 individual noncontact dispensing heads. The HD-NAPPA slides were stored under an argon gas filled container at room temperature until the day of use when proteins were expressed.

Protein Expression on M-HD-NAPPA.

Arrays were blocked with SuperBlock (Thermo Fisher Scientific, Rockford, Ill.) prior to expression to reduce nonspecific binding, rinsed with DI water and centrifuged dry. The nano-wells were filled with human cell-free expression system (In Vitro Transcription and Translation coupled system; IVTT; Thermo Fisher Scientific) and a custom micro-reactor device was used for the protein expression. After sealing the wells with a polystyrene membrane under 200 PSI pressure, the reactor was incubated for 2 h at 30° C. for expression and for 0.5 h at 15° C. for protein capture, followed by blocking with 5% skim milk in phosphate buffered saline with 0.2% tween 20 (PBST) for 30 min. Anti-GST murine monoclonal Ab (mAb; Cell signaling technology, Danvers, Mass.) was used to assess protein display followed by detection with Alexa 647-labeled Goat antimouse IgG (H+L) secondary Ab (A-21235, Thermo Fisher Scientific).

Subjects and Samples.

Serum samples were obtained in cross-sectional studies from patients with Mtb culture-proven TB before or within the first 7 days of antituberculous treatment initiation and from asymptomatic controls (Table 5). Subjects were enrolled in two different settings, in public hospitals in New York City, United States, and at Edendale Hospital in KwaZulu-Natal, South Africa (SA). Subjects provided informed written consent prior to enrollment and blood draw. Serum was obtained by collecting peripheral venous blood into BD Vacutainer Serum Separation Tubes (SST; Becton, Dickinson and Company, New Jersey) that do not contain any additives. Within 1-3 h after blood draw the samples were centrifuged at room temperature for 10 mins at 3000 rpm and serum was aliquoted and stored at −80° C. until further use.

TABLE 5
Demographics and clinical characteristics of TB patients and controls.

| | US | | | | SA | | | |
|---|---|---|---|---|---|---|---|---|
| | TB patients (n = 66) | | Controls (n = 68) | | TB patients (n = 102) | | Controls (n = 49) | |
| Age, median (range) | 36 | (20-70) | 42 | (22-67) | 33 | (23-42) | 35 | (25-53) |
| Male sex, n (%) | 50 | (75) | 31 | (45) | 50 | (49) | 12 | (25) |
| Non-US born^{a}, n (%) | 59 | (89) | 23 | (34) | NA | | NA | |
| TST positive (%) | NA | | (45) | | NA | | NA | |
| AFB smear positive, n (%) | 36 | (54) | NA | | 79 | (77) | NA | |
| HIV-infected, n (%) | 21 | (32) | 46 | (68) | 89 | (87) | 24 | (49) |
| CD4, median cells/mm³ (range) | 150 | (121-271) | 539 | (11-1541) | 199 | (0-1000) | 602 | (374-1237) |

^{a}Subjects emigrated from various TB endemic regions, including Asia, South America and Africa;
TST: Tuberculin skin-test;
AFB: acid fast bacilli.

The studies were approved by the Institutional Review Boards of Arizona State University; the Albert Einstein College of Medicine, New York; and the University of KwaZulu-Natal, SA. The samples were divided into four subgroups according to the region (US, SA) and HIV status (HIV+/HI−; Table 1). Prior to performing assays, the samples in each subgroup were randomized into two even sets: one set for performing the screening/deconvolution array and one independent set for performing the validation array (Table 1).

M-HD-NAPPA—Concept Validation.

In order to evaluate the M-HD-NAPPA array screening workflow, 96 Mtb genes were selected from initial individual gene glass-slide NAPPA results (data not shown but available upon request) and scientific literature to create a gene set to validate immunodetection of individual proteins within a triple protein mix. In addition, 288 Mtb clones were randomly selected and printed those as individual genes as well as triple gene mixes on the HD-NAPPA slides. Ab binding was performed with a pooled sample set from 3 HIV−, TB+ subjects that had documented Ab reactivity to various proteins from prior studies as well as mAb anti-GST for protein display level. During scanning of the silicon slides, the scanner parameters were adjusted to focus the signal detection located within the wells, which were 75 μm deeper than flat glass slide based flat glass NAPPA.

M-HD-NAPPA—Discovery Screen.

A multiplex Mtb array was created containing all 4045 genes, spread among 1431 multiplexed Mtb gene spots along with the 96 individual Mtb genes and 7 viral single gene controls. There were four identical subarrays printed on each of the M-HD-NAPPA slides.

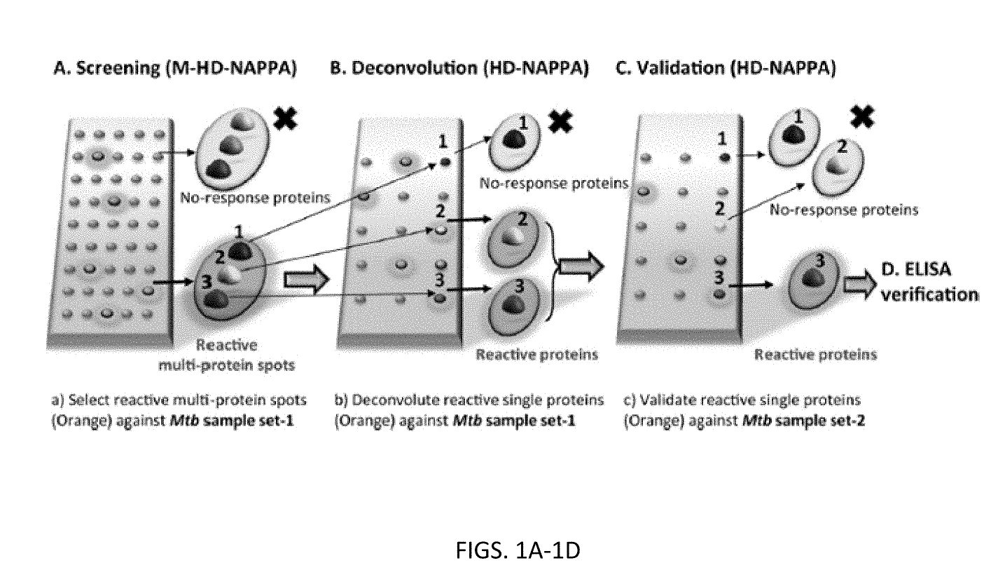

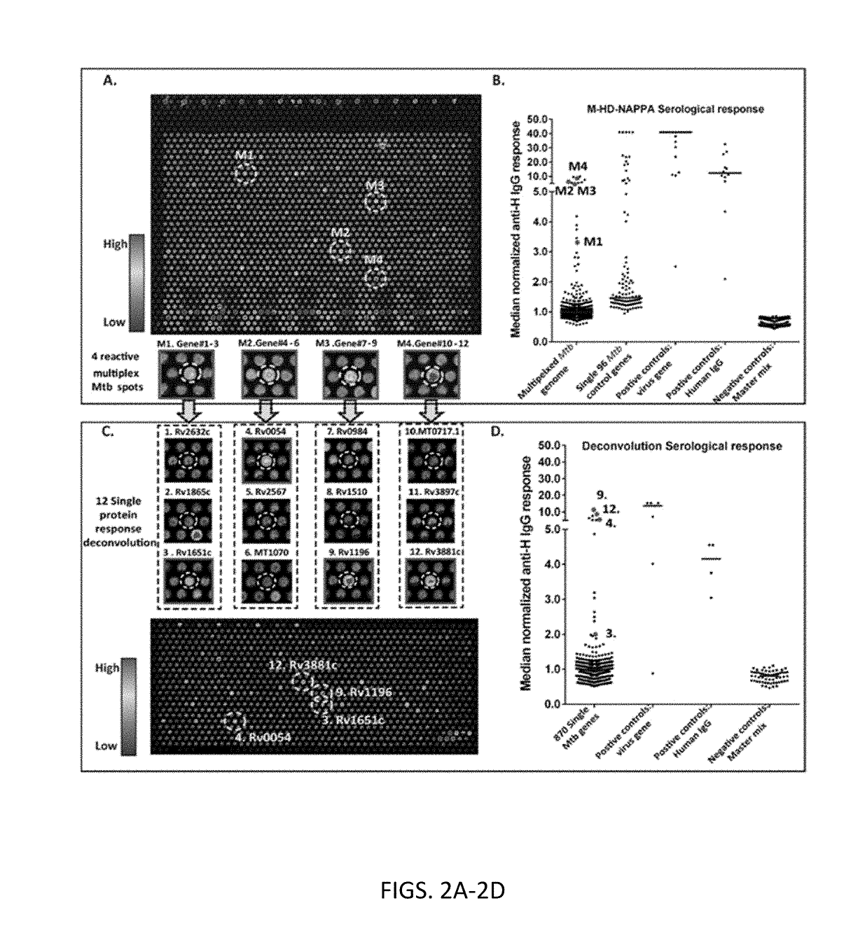

M-HD-NAPPA arrays were expressed for probing against 120 subject samples (Table 1) to identify Mtb antibody binding proteins (FIG. 1A). A four well gasket was utilized to create a chamber around each subarray (ProPlate Multi-well chamber, GraceBio-Labs, Bend, Oreg.) and placed 650 μl of individual serum sample, diluted to 1:150 in 5% skim milk in PBST in each chamber, which was sealed with an opposing glass slide. This was then incubated overnight (14-16 h) at 4° C. with gentle shaking to ensure even exposure of array surface to sample. The arrays were then rinsed with 5% skim milk/PB ST and Ab binding detected with Alex647 labeled Goat anti-human IgG (H+L) and 1:200 diluted Cy3 labeled Goat anti-human IgA (Jackson ImmunoResearch Labs, West Grove, Pa.). The slides were rinsed again to remove unbound secondary Ab, dried by centrifugation and scanned at 635 nm and 535 nm with Tecan PowerScanner. The resulting images were quantified with the ArrayPro Analyzer Software (Media Cybernetics, Inc.). Data were extracted and median normalized within each subarray. To assure a sufficient margin between positive and negative Ab reactivity a signal cutoff of 1.4 was used to identify spots for further deconvolution with the individual gene HD-NAPPA. In addition, the sensitivity and specificity were calculated within subgroups and all sample combinations. Those protein targets showing higher than 10% sensitivity in any of the four subgroups or higher than 5% sensitivity in all combined groups were selected for deconvolution.

FIG. 1 is a schematic strategy of Mtb biomarker discovery, as described herein. FIG. 1A describes the screening stage: samples (set-1) were probed against 1431 Mtb 3*gene spots (4045 individual Mtb genes) multiplex HD-NAPPA array and multiplex spots showing response with TB+ sera were selected for deconvolution. FIG. 1B described the deconvolution stage: the same sample sets were probed against 870 single Mtb gene HD-NAPPA arrays. FIG. 1C describes the validation stage: independent samples (set-2) were probed against single Mtb genes identified in the deconvolution using the HD-NAPPA array. FIG. 1D describes the verification stage: both sample sets 1 and 2 were probed against the 8 most promising biomarker Mtb genes in Rapid antigenic protein in situ display (RAPID) ELISA for verification of their performance.

HD-NAPPA—Deconvolution.

Overall, 272 multiplex spots (792 single genes) were calculated showing differential responses between TB positive and negative subgroups. These 792 genes were printed as single genes on the HD-NAPPA. In addition, initial individual 96 Mtb genes were included (from unpublished prior studies and the literature) that were not among the 792 genes (62 genes overlapped) and the controls resulting in a final 870 single gene HD-NAPPA of which 8 subarrays fitted on each slide. To identify the specific protein targets, the same subject samples used for the M-HD-NAPPA screen were tested and the slides were processed as described earlier (FIG. 1B). Those genes with greater than 10% sensitivity in any of the four subgroups were selected as candidates for further analysis.

HD-NAPPA—Validation.

Individual HD-NAPPA arrays as described above were created for deconvolution as well as validation with biologically independent sample sets (n=124; FIG. 1C). Each sample was tested on an independent array. These samples were randomly assigned to processing days so as to minimize any day-to-day processing bias. Array processing was performed as described above.

RAPID ELISA.

Rapid antigenic protein in situ display (RAPID) ELISA was used as described to verify the selected candidate proteins according to the three criteria described above (FIG. 1D). 285 individual subject samples were used (1:500 dilution) from both the discovery and validation array experiments (Table 1) to assess binding to selected proteins. As a negative control, the Mtb protein Rv1553 was used, which showed a normalized response close to one (0.972 0.079) on prior array analyses. Binding was detected using SuperSignal West Femto Chemiluminescent Substrate (Thermo Fisher Scientific). The chemiluminescent signal was measured using the EnVision 2104 Multilabel Reader (PerkinElmer, San Jose, Calif.) at 460 nm with 0.1 s per well, for a total of 10 repeat reads for each plate. The average from the last five reads was used for analysis. The Ab reactivity data were normalized by subtracting the reactivity to the negative control protein tested on the same run. Normalized values were log₁₀ transformed and responses below background were set to zero. Normalized and transformed reactivity from replicate protein runs were then averaged for each sample.

HD-NAPP A—Validation.

A visual inspection of each array image was conducted and spot by spot to avoid artifacts. The data were median normalized, and the sensitivity and specificity were calculated at cutoff 1.4. The odd's ratio of a positive response was calculated using Firth's penalized likelihood logistic regression. Finally, the area under the receiver operator characteristic (ROC) curve (AUC) was calculated, which is a measure of marker performance across a range of cutoff values. It was set as 0.55, which elucidated the antigens likely to be positive in the TB groups. Only those genes that passed deconvolution and validation with the second set of samples were taken as possible biomarker candidates.

Because of the high level of heterogeneity of responses within the subject subcategories, an analysis was performed of the candidate biomarkers with the deconvolution and validation array data combined. Briefly, the normalized data of the deconvolution and validation within each subgroups were combined as 4 paired subgroups and processed with the same criteria as the validation array analysis. Those genes with a sensitivity higher than 20%, an odd's ratio >1.5 and an AUC value >0.55 in the combined analysis were selected as the biomarkers for ELISA verification testing.

ELISA.

ROC curve analysis was used to assess the performance of each protein tested via ELISA for discriminating TB positive from TB negative patients in each of the four patient subgroups. The pROC R package was used to conduct the analysis. For each protein, several ROC statistics were measured including AUC, the sensitivity at 80% specificity and the specificity at 80% sensitivity. The p value was calculated for the Wilcoxon rank sum test of no difference between the TB positive and TB negative patients. P values were not adjusted for multiple testing and should not be interpreted as strict statistical p values because of the protein selection process and sample re-use. Multiprotein panels were developed to classify TB positive and TB negative patients in each subgroup. The classifier for each subgroup was a logistic regression model. All possible logistic regression models were evaluated using the Bayes Information Criteria (BIC) to identify the best set of proteins for each subgroup. This analysis was conducted using the bestglm R package and Morgan-Tatar search. For each sample the fitted (noncalibrated) probability of TB positivity was calculated. This probability was calculated using leave-one-out cross validation. ROC curves were generated using both the fitted and cross-validated probabilities, and calculated ROC statistics including the AUC, the specificity at 80% sensitivity and the sensitivity at 80% specificity.

Various features and advantages of the invention are set forth in the following claims.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

For reasons of completeness, various aspects of the invention are set out in the following numbered clauses, as well as the following claims:

Clause 1. A method of diagnosing a subject as having tuberculosis (TB), the method comprising: performing an assay on a biological sample obtained from a subject; and measuring or detecting at least one TB biomarker or fragment thereof selected from the group consisting of Rv0054 (ssb), Rv0813c, Rv2031c, (HspX/acr), Rv0222 (echA1), Rv0948c, Rv2853 (PE_PGRS48), Rv3405c, and Rv3544c (fadE28); wherein the measurement or detection of the at least one TB biomarker or fragment thereof indicates that the subject has a TB infection.

Clause 2. The method of clause 1, wherein the biological sample is at least one of whole blood, serum, plasma, urine, saliva, sweat, sputum, semen, mucus, lacrimal fluid, lymph fluid, amniotic fluid, interstitial fluid, lung lavage, cerebrospinal fluid, and feces.

Clause 3. The method of clause 1 or 2, wherein the assay is an immunoassay.

Clause 4. The method of any of clauses 1-3, wherein the immunoassay has a sensitivity of at least 80.0% and a specificity of at least 50.0%.

Clause 5. The method of any of clauses 1-4, wherein the immunoassay has a specificity of at least 80.0% and a sensitivity of at least 50.0%.

Clause 6. The method of any of clauses 1-5, wherein the at least one TB biomarker or fragment thereof is selected from the group consisting of Rv0831c, Rv2031c, (HspX/acr).

Clause 7. The method of any of clauses 1-6, wherein the at least one TB biomarker or fragment thereof is selected from the group consisting of Rv0054 and Rv0948c.

Clause 8. The method of any of clauses 1-7, wherein the at least one TB biomarker or fragment thereof is selected from the group consisting of Rv2853, Rv0054, Rv0831c, Rv3544c, and Rv0222.

Clause 9. The method of any of clauses 1-8, wherein the at least one TB biomarker or fragment thereof is Rv3405c.

Clause 10. The method of any of clauses 1-9, wherein the at least one TB biomarker or fragment thereof is selected from the group consisting of Rv0948c, Rv2853, Rv3405c, and Rv3544c.

Clause 11. The method of any of clauses 1-10, wherein the at least one TB biomarker or fragment thereof is selected from the group consisting of Rv0054, Rv2853, and Rv3405c.

Clause 12. A panel of biomarkers for diagnosing a subject as having tuberculosis (TB), the panel comprising: at least one TB biomarker or fragment thereof selected from the group consisting of Rv0054 (ssb), Rv0813c, Rv2031c, (HspX/acr), Rv0222 (echA1), Rv0948c, Rv2853 (PE_PGRS48), Rv3405c, and Rv3544c (fadE28); wherein the measurement or detection of the at least one TB biomarker or fragment thereof indicates that the subject has a TB infection.

Clause 13. The panel of clause 12, wherein the at least one TB biomarker or fragment thereof is measured or detected using an immunoassay.

Clause 14. The panel of clause 12 or 13, wherein the immunoassay has a sensitivity of at least 80.0% and a specificity of at least 50.0%.

Clause 15. The panel of any of clauses 12-14, wherein the immunoassay has a specificity of at least 80.0% and a sensitivity of at least 50.0%.

Clause 16. The panel of any of clauses 12-15, wherein the at least one TB biomarker or fragment thereof is selected from the group consisting of Rv0831c, Rv2031c, (HspX/acr).

Clause 17. The panel of any of clauses 12-16, wherein the at least one TB biomarker or fragment thereof is selected from the group consisting of Rv0054 and Rv0948c.

Clause 18. The panel of any of clauses 12-17, wherein the at least one TB biomarker or fragment thereof is selected from the group consisting of Rv0054 and Rv0948c.

Clause 19. The panel of any of clauses 12-18, wherein the at least one TB biomarker or fragment thereof is selected from the group consisting of Rv2853, Rv0054, Rv0831c, Rv3544c, and Rv0222.

Clause 20. The panel of any of clauses 12-19, wherein the at least one TB biomarker or fragment thereof is Rv3405c.

Clause 21. The panel of any of clauses 12-20, wherein the at least one TB biomarker or fragment thereof is selected from the group consisting of Rv0948c, Rv2853, Rv3405c, and Rv3544c.

Clause 22. The panel of any of clauses 12-21, wherein the at least one TB biomarker or fragment thereof is selected from the group consisting of Rv0054, Rv2853, and Rv3405c.

What is claimed is:

1. A method of diagnosing a subject as having tuberculosis (TB), the method comprising:
performing an assay on a biological sample obtained from a subject; and
measuring or detecting at least one TB biomarker or fragment thereof selected from the group consisting of Rv0054 (ssb), Rv0813c, Rv2031c, (HspX/acr), Rv0222 (echA1), Rv0948c, Rv2853 (PE_PGRS48), Rv3405c, and Rv3544c (fadE28);
wherein the measurement or detection of the at least one TB biomarker or fragment thereof indicates that the subject has a TB infection.

2. The method of claim 1, wherein the biological sample is at least one of whole blood, serum, plasma, urine, saliva, sweat, sputum, semen, mucus, lacrimal fluid, lymph fluid, amniotic fluid, interstitial fluid, lung lavage, cerebrospinal fluid, and feces.

3. The method of claim 1, wherein the assay is an immunoassay.

4. The method of claim 3, wherein the immunoassay has:
a sensitivity of at least 80.0% and a specificity of at least 50.0%; or
a specificity of at least 80.0% and a sensitivity of at least 50.0%.

5. The method of claim 1, wherein the at least one TB biomarker or fragment thereof is selected from the group consisting of Rv0831c, Rv2031c, (HspX/acr), and Rv0948c.

6. The method of claim 1, wherein the at least one TB biomarker or fragment thereof is selected from the group consisting of Rv0054 and Rv0948c.

7. The method of claim 1, wherein the at least one TB biomarker or fragment thereof is selected from the group consisting of Rv2853, Rv0054, Rv0831c, Rv3544c, and Rv0222.

8. The method of claim 1, wherein the at least one TB biomarker or fragment thereof is Rv3405c.

9. The method of claim 1, wherein the at least one TB biomarker or fragment thereof is selected from the group consisting of Rv0948c, Rv2853, Rv3405c, and Rv3544c.

10. The method of claim 1, wherein the at least one TB biomarker or fragment thereof is selected from the group consisting of Rv0054, Rv2853, and Rv3405c.

11. A panel of biomarkers for diagnosing a subject as having tuberculosis (TB), the panel comprising:
at least one TB biomarker or fragment thereof selected from the group consisting of Rv0054 (ssb), Rv0813c, Rv2031c, (HspX/acr), Rv0222 (echA1), Rv0948c, Rv2853 (PE_PGRS48), Rv3405c, and Rv3544c (fadE28);
wherein the measurement or detection of the at least one TB biomarker or fragment thereof indicates that the subject has a TB infection.

12. The panel of claim 11, wherein the at least one TB biomarker or fragment thereof is measured or detected in a biological sample from the subject, and wherein the biological sample is at least one of whole blood, serum, plasma, urine, saliva, sweat, sputum, semen, mucus, lacrimal fluid, lymph fluid, amniotic fluid, interstitial fluid, lung lavage, cerebrospinal fluid, and feces.

13. The panel of claim 11, wherein the at least one TB biomarker or fragment thereof is measured or detected using an immunoassay.

14. The panel of claim 13, wherein the immunoassay has:
a sensitivity of at least 80.0% and a specificity of at least 50.0%, or
a specificity of at least 80.0% and a sensitivity of at least 50.0%.

15. The panel of claim 11, wherein the at least one TB biomarker or fragment thereof is selected from the group consisting of Rv0831c, Rv2031c, (HspX/acr), and Rv0948c.

16. The panel of claim 11, wherein the at least one TB biomarker or fragment thereof is selected from the group consisting of Rv0054 and Rv0948c.

17. The panel of claim 11, wherein the at least one TB biomarker or fragment thereof is selected from the group consisting of Rv2853, Rv0054, Rv0831c, Rv3544c, and Rv0222.

18. The panel of claim 11, wherein the at least one TB biomarker or fragment thereof is Rv3405c.

19. The panel of claim 11, wherein the at least one TB biomarker or fragment thereof is selected from the group consisting of Rv0948c, Rv2853, Rv3405c, and Rv3544c.

20. The panel of claim 11, wherein the at least one TB biomarker or fragment thereof is selected from the group consisting of Rv0054, Rv2853, and Rv3405c.
